(19) United States
(12) Patent Application Publication
(10) Pub. No.: US 2026/0351349 A1
(43) Pub. Date: Nov. 5, 2026

(54) MACHINE LEARNING ENABLED ESTIMATION OF DIFFERENCES BETWEEN PROTEIN SEQUENCES

(71) Applicants: Genentech, Inc., South San Francisco, CA (US); Hoffmann-La Roche Inc., Little Falls, NJ (US)

(72) Inventors: Andrew Philip LEAVER-FAY, Chapel Hill, NC (US); Edith LEE, South San Francisco, CA (US); Wei-Ching LIANG, South San Francisco, CA (US); Yao-Yu LIN, Brooklyn, NY (US); Stefania VASILAKI, Zurich (CH); Pedro Henrique OLIVEIRA PINHEIRO, Brooklyn, NY (US); Natasa TAGASOVSKA, Vaud (CH); Andrew Martin WATKINS, San Francisco, CA (US); Kyunghyun CHO, New York, NY (US); Nathan Christopher FREY, New York, NY (US); Vladimir GLIGORIJEVIC, New Brunswick, NJ (US)

(21) Appl. No.: 19/639,714

(22) Filed: Apr. 6, 2026

Related U.S. Application Data
(63) Continuation of application No. PCT/US2024/050017, filed on Oct. 4, 2024.
(60) Provisional application No. 63/588,671, filed on Oct. 6, 2023.

(51) Int. Cl. G16B 40/00; G16B 20/20; G16B 30/00

(52) U.S. Cl. CPC G16B 40/00; G16B 20/20; G16B 30/00

(57) ABSTRACT

A method may include generating a training dataset including a pair of sample molecules. The sample molecule pair includes a first molecule and a second molecule. A property computation model is trained to determining a sequence difference comprising a difference between an amino acid sequence of the first molecule and an amino acid sequence of the second molecule. The property computation model is further trained to generate a relative embedding representative of the sequence difference and associating the sequence difference with the sample molecule pair. The property computation model is further trained to determine, based on the relative embedding, a property difference corresponding to a difference between a value of the property exhibited by the first molecule and a value of the property exhibited the second molecule. The trained property computation model may be applied to determine a property difference of a pair of input molecules.

### CROSS REFERENCE TO RELATED APPLICATION

This application claims priority to U.S. Provisional Application No. 63/588,671, entitled “MACHINE LEARNING ENABLED ESTIMATION OF DIFFERENCES BETWEEN PROTEIN SEQUENCES” and filed on Oct. 6, 2023, the disclosure of which is incorporated herein by reference in its entirety.

### TECHNICAL FIELD

The subject matter described herein relates generally to molecular design, and more specifically, to a computation model for determining the difference in the property exhibited by two different protein molecules and techniques for leveraging the computation model to enhance molecular properties.

### INTRODUCTION

A molecule is a group of two more atoms held together by chemical bonds. Molecules form the smallest identifiable unit into which a pure substance can be divided while still retaining the composition and chemical properties of that substance. Various properties of a molecule, including its ability to function as a therapeutic, may be contingent upon its composition and conformation (or three-dimensional structure). Large molecules (also known as biopharmaceuticals, biologicals, or biologics) are molecules ranging between approximately 3000 Daltons and 150,000 Daltons in molecular weight. Large molecule drugs are often derivatives of natural human proteins, which modulate many essential cellular functions such as enzymatic reactions, transport of molecules, regulation and execution of a number of biological pathways, cell growth, proliferation, nutrient uptake, morphology, motility, intercellular communication, and/or the like. Examples of therapeutic proteins include antibodies, chimeric antigen receptors (CARs), enzymes, hormones, cytokines, and/or the like. A single large molecule can have more than 1,300 amino acid residues, which are linked by peptide bonds to form one or more polypeptide. Due to their size and complexity, large molecule drugs are recombinantly produced by engineered cells instead of being chemically synthesized like the majority of small molecule drugs. Moreover, large molecule therapeutics are usually delivered through injection or infusion due to the ineffectiveness of oral administration. The development of a large molecule drug may entail designing one or more sequences of amino acid residues capable of binding to a target (e.g., a protein, a nucleic acid, and/or the like) with sufficient specificity and absent undesirable traits such as immunogenicity, self-association, instability, and/or the like.

### SUMMARY

Systems, methods, and articles of manufacture, including computer program products, are provided for machine learning enabled estimation of differences between protein molecules. In one aspect, there is provided a system that includes at least one memory and at least one data processor. The at least one memory may store instructions that result in operations when executed by the at least one processor. The operations may include: generating a training dataset including a pair of sample molecules, the sample molecule pair including a first molecule and a second molecule; training, based at least on the training dataset, a property computation model, wherein the property computation model is trained to at least, determine a sequence difference between the first molecule and second molecule, the sequence difference comprising a difference between an amino acid sequence of the first molecule and an amino acid sequence of the second molecule, generate a relative embedding representative of the sequence difference and associating the sequence difference with the sample molecule pair, and determine, based at least on the relative embedding of the sample molecule pair, a property difference for the sample molecule pair, where the property difference for the molecule pair corresponds to a difference between a value of the property exhibited by the first molecule and a value of the property exhibited the second molecule; receiving a pair of input molecules; and applying the property computation model to determine a property difference of the pair of input molecules.

In another aspect, there is provided a computer-implemented method for machine learning enabled estimation of differences between protein molecules. The method may include: generating a training dataset including a pair of sample molecules, the sample molecule pair including a first molecule and a second molecule; training, based at least on the training dataset, a property computation model, wherein the property computation model is trained to at least, determine a sequence difference between the first molecule and second molecule, the sequence difference comprising a difference between an amino acid sequence of the first molecule and an amino acid sequence of the second molecule, generate a relative embedding representative of the sequence difference and associating the sequence difference with the sample molecule pair, and determine, based at least on the relative embedding of the sample molecule pair, a property difference for the sample molecule pair, where the property difference for the molecule pair corresponds to a difference between a value of the property exhibited by the first molecule and a value of the property exhibited the second molecule; receiving a pair of input molecules; and applying the property computation model to determine a property difference of the pair of input molecules.

In another aspect, there is provided a computer program product including a non-transitory computer readable medium storing instructions. The instructions may cause operations may executed by at least one data processor. The operations may include: generating a training dataset including a pair of sample molecules, the sample molecule pair including a first molecule and a second molecule; training, based at least on the training dataset, a property computation model, wherein the property computation model is trained to at least, determine a sequence difference between the first molecule and second molecule, the sequence difference comprising a difference between an amino acid sequence of the first molecule and an amino acid sequence of the second molecule, generate a relative embedding representative of the sequence difference and associating the sequence difference with the sample molecule pair, and determine, based at least on the relative embedding of the sample molecule pair, a property difference for the sample molecule pair, where the property difference for the molecule pair corresponds to a difference between a value of the property exhibited by the first molecule and a value of the property exhibited the second molecule; receiving a pair of input molecules; and applying the property computation model to determine a property difference of the pair of input molecules.

In some variations, one or more features disclosed herein including the following features can optionally be included in any feasible combination.

In some variations, the property includes expression, affinity, specificity, developability, or biological activity.

In some variations, the sample molecule pair is generated by at least pairing sample molecules from a set of sample molecules having known values for the property.

In some variations, the generating the training dataset includes pairing a first sample molecule and a second sample molecule from a set of sample molecules to generate the sample molecule pair, and pairing the first sample molecule with a third sample molecule from the set of sample molecules to generate another sample molecule pair.

In some variations, the property computation model includes a language model, and the training the property computation model includes training the language model to generate the relative embedding of the sample molecule pair.

In some variations, the language model is trained to at least generate, for the sample molecule pair, a first embedding for the first sample molecule and a second embedding for the second sample molecule, and generate the relative embedding of each sample molecule pair by at least determining a difference between the first embedding and the second embedding.

In some variations, the difference between the first embedding and the second embedding corresponds to the sequence difference comprising the difference between the amino acid sequence of the first molecule and the amino acid sequence of the second molecule.

In some variations, the property computation model further includes a neural network coupled with the language model, and the training the property computation model includes training the neural network to determine, based at least on the relative embedding of each sample molecule pair, the property difference for each sample molecule pair.

In some variations, one or more mutations associated with an improvement to the property are identified based at least on the property difference of the pair of input molecules.

In some variations, the one or more mutations include a point mutation where the pair of input molecules differ by a single position in each corresponding amino acid sequence.

In some variations, the one or more mutations include a combination of multiple point mutations where the pair of input molecules differ by multiple positions in each corresponding amino acid sequence.

In some variations, each sample molecule includes an antibody or a portion of the antibody.

In some variations, each sample molecule includes a variable region, an antigen binding region, a heavy chain, and/or a light chain of an antibody.

In another aspect, there is provided a system for identifying a mutation. The system may include at least one memory and at least one data processor. The at least one memory may store instructions that result in operations when executed by the at least one processor. The operations may include: applying a property computation model to determine a difference in a value of a property exhibited by a first input molecule and a value of the property exhibited by a second input molecule; determining an amino acid sequence of the first input molecule and an amino acid sequence of the second input molecule; identifying a first mutation by comparing the amino acid sequence of the first input molecule to the amino acid sequence of the second input molecule; and identifying, based at least on the difference in the value of the property exhibited by the first input molecule and the second input molecule, that the first mutation improves the value of the property.

In one aspect, there is provided a computer-implemented method for identifying a mutation. The method may include: applying a property computation model to determine a difference in a value of a property exhibited by a first input molecule and a value of the property exhibited by a second input molecule; determining an amino acid sequence of the first input molecule and an amino acid sequence of the second input molecule; identifying a first mutation by comparing the amino acid sequence of the first input molecule to the amino acid sequence of the second input molecule; and identifying, based at least on the difference in the value of the property exhibited by the first input molecule and the second input molecule, that the first mutation improves the value of the property.

In another aspect, there is provided a computer program product including a non-transitory computer readable medium storing instructions. The instructions may cause operations to be executed by at least one data processor. The operations may include: applying a property computation model to determine a difference in a value of a property exhibited by a first input molecule and a value of the property exhibited by a second input molecule; determining an amino acid sequence of the first input molecule and an amino acid sequence of the second input molecule; identifying a first mutation by comparing the amino acid sequence of the first input molecule to the amino acid sequence of the second input molecule; and identifying, based at least on the difference in the value of the property exhibited by the first input molecule and the second input molecule, that the first mutation improves the value of the property.

In some variations, one or more features disclosed herein including the following features can optionally be included in any feasible combination.

In some variations, the property includes expression, affinity, specificity, developability, or biological activity.

In some variations, the property computation model is applied to determine the difference in the value of the property exhibited by the first input molecule and a third input molecule. An amino acid sequence of the third molecule is determined. A second mutation is identified by at least comparing the amino acid sequence of the first input molecule to the amino acid sequence of the third input molecule. Identifying the second mutation as improving the value of the property based at least on the difference in the value of the property exhibited by the first input molecule and the third input molecule.

In some variations, the first mutation includes a type of amino acid residue occupying a first position in the amino acid sequence of the first input molecule, and the second mutation includes a same type of amino acid residue occupying a second position in the amino acid sequence of the first input molecule.

In some variations, the first mutation includes a position in the amino acid sequence of the first input molecule being occupied by a first type of amino acid residue, and the second mutation includes a same position in the amino acid sequence of the first input molecule being occupied by a second type of amino acid residue.

In some variations, at least one output molecule exhibiting the first mutation and/or the second mutation is generated.

In some variations, each input molecule includes an antibody or a portion of the antibody.

In some variations, each sample molecule includes a variable region, an antigen binding region, a heavy chain, and/or a light chain of an antibody.

In another aspect, there is provided a system for identifying a combination of mutations. The system may include at least one memory and at least one data processor. The at least one memory may store instructions that result in operations when executed by the at least one processor. The operations may include: determining a plurality of point mutations, where each point mutation of the plurality of point mutations is associated with an improvement to a value of a property; generating a plurality of mutation combinations, where each mutation combination of the plurality of mutation combinations includes point mutations selected from the plurality of point mutations; generating a plurality of input molecules, where each input molecule of the plurality of input molecules exhibits one mutation combination from the plurality of mutation combinations; applying a property computation model that has been trained to determine a difference in between a value of the property exhibited by two input molecules from the plurality of input molecules; and identifying, based at least on the difference between the value of the property exhibited by the two input molecules from the plurality of input molecules, at least one mutation combination as being associated with the improvement to the value of the property.

In another aspect, there is provided a computer-implemented method for identifying a combination of mutations. The method may include: determining a plurality of point mutations, where each point mutation of the plurality of point mutations is associated with an improvement to a value of a property; generating a plurality of mutation combinations, where each mutation combination of the plurality of mutation combinations includes point mutations selected from the plurality of point mutations; generating a plurality of input molecules, where each input molecule of the plurality of input molecules exhibits one mutation combination from the plurality of mutation combinations; applying a property computation model that has been trained to determine a difference in between a value of the property exhibited by two input molecules from the plurality of input molecules; and identifying, based at least on the difference between the value of the property exhibited by the two input molecules from the plurality of input molecules, at least one mutation combination as being associated with the improvement to the value of the property.

In another aspect, there is provided a computer program product including a non-transitory computer readable medium storing instructions. The instructions may cause operations may executed by at least one data processor. The operations may include: determining a plurality of point mutations, where each point mutation of the plurality of point mutations is associated with an improvement to a value of a property; generating a plurality of mutation combinations, where each mutation combination of the plurality of mutation combinations includes point mutations selected from the plurality of point mutations; generating a plurality of input molecules, where each input molecule of the plurality of input molecules exhibits one mutation combination from the plurality of mutation combinations; applying a property computation model that has been trained to determine a difference in between a value of the property exhibited by two input molecules from the plurality of input molecules; and identifying, based at least on the difference between the value of the property exhibited by the two input molecules from the plurality of input molecules, at least one mutation combination as being associated with the improvement to the value of the property.

In some variations, one or more features disclosed herein including the following features can optionally be included in any feasible combination.

In some variations, each input molecule is generated by applying the one mutation combination to an amino acid sequence of a selected molecule.

In some variations, the property includes expression, affinity, specificity, developability, or biological activity.

In another aspect, there is provided a system for machine learning enabled enhancement of a molecular property. The system may include at least one memory and at least one data processor. The at least one memory may store instructions that result in operations when executed by the at least one processor. The operations may include: identifying a first pair of input molecules including a first input molecule and a second input molecule, where the first input molecule exhibits a mutation relative to the second input molecule; applying a property computation model that has been trained to determine, based at least on a first amino acid sequence of the first input molecule and a second amino acid sequence of the second input molecule, a difference between a first value of a property exhibited by the first input molecule and a second value of the property exhibited by the second input molecule; identifying, based at least on the difference between the first value and the second value of the property, the mutation as improving to the property; and generating a candidate molecule by at least modifying a selected molecule to include the mutation.

In another aspect, there is provided a computer-implemented method for machine learning enabled enhancement of a molecular property. The method may include: identifying a first pair of input molecules including a first input molecule and a second input molecule, where the first input molecule exhibits a mutation relative to the second input molecule; applying a property computation model that has been trained to determine, based at least on a first amino acid sequence of the first input molecule and a second amino acid sequence of the second input molecule, a difference between a first value of a property exhibited by the first input molecule and a second value of the property exhibited by the second input molecule; identifying, based at least on the difference between the first value and the second value of the property, the mutation as improving to the property; and generating a candidate molecule by at least modifying a selected molecule to include the mutation.

In another aspect, there is provided a computer program product including a non-transitory computer readable medium storing instructions. The instructions may cause operations may executed by at least one data processor. The operations may include: identifying a first pair of input molecules including a first input molecule and a second input molecule, where the first input molecule exhibits a mutation relative to the second input molecule; applying a property computation model that has been trained to determine, based at least on a first amino acid sequence of the first input molecule and a second amino acid sequence of the second input molecule, a difference between a first value of a property exhibited by the first input molecule and a second value of the property exhibited by the second input molecule; identifying, based at least on the difference between the first value and the second value of the property, the mutation as improving to the property; and generating a candidate molecule by at least modifying a selected molecule to include the mutation.

In some variations, one or more features disclosed herein including the following features can optionally be included in any feasible combination.

In some variations, a second pair of input molecules including the first input molecule and a third input molecule is identified. The first input molecule exhibits another mutation relative to the third input molecule. The property computation model is applied to determine, based at least on the first amino acid sequence of the first input molecule and a third amino acid sequence of the third input molecule, a difference between the first value of the property exhibited by the first input molecule and a third value of the property exhibited by the third input molecule. The first pair of input molecules is identified to include the first input molecule instead of the third input molecule based at least on the difference between the first value and the third value of the property indicating that the first input molecule exhibits a better value for the property than the third input molecule.

In some variations, the mutation includes a plurality of point mutations.

In some variations, the plurality of point mutations is identified by at least identifying each individual point mutation as improving the value of the property.

In some variations, the identifying each individual point mutation as improving the value of the property includes generating a plurality of pairs of input molecules, where each pair of input molecules includes two input molecules whose amino acid sequences differ by a single point mutation; applying the property computation model to determine, for each pair of input molecules, one input molecule exhibiting a better value for the property than the other input molecule; and identifying, for each pair of input molecule, a point mutation that is exhibited by the one input molecule exhibiting the better value for the property as one of the plurality of point mutations.

In some variations, the single point mutation includes one amino acid sequence including a first type of amino acid residue at a particular position and another amino acid sequence including a second type of amino acid residue at a same position.

In some variations, the single point mutation includes one amino acid sequence including a particular type of amino acid residue at a first position and another amino acid sequence including a same type of amino acid residue at a second position.

In some variations, the property computation model includes a language model that has been trained to generate a relative embedding representative of a difference between a first amino acid sequence of the first input molecule and a second amino acid sequence of the second input molecule.

In some variations, the property computation model further includes a neural network coupled with the language model, and the neural network has been trained to determine, based at least on the relative embedding, the difference between the first value and the second value of the property.

In some variations, each of the first input molecule and the second input molecule includes an antibody.

In some variations, each of the first input molecule and the second input molecule includes a portion of an antibody.

In some variations, each of the first input molecule and the second input molecule includes a variable region, an antigen binding region, a heavy chain, and/or a light chain of an antibody.

In some variations, the property includes expression, affinity, specificity, developability, or biological activity.

In another aspect, there is provided a system for training a property computation model. The system may include at least one memory and at least one data processor. The at least one memory may store instructions that result in operations when executed by the at least one processor. The operations may include: identifying a set of sample molecules, where each sample molecule in the set of sample molecules is associated with a respective value for a property; generating a plurality of sample molecule pairs for inclusion in a training dataset, where the generating the plurality of sample molecule pairs includes pairing a first sample molecule and a second sample molecule selected from the set of sample molecules to generate a first sample molecule pair, and pairing the first sample molecule and a third sample molecule selected from the set of sample molecules to generate a second sample molecule pair; and training, based at least on the training dataset, a property computation model to determine, based at least on a sequence difference between two input molecules, a property difference between the two input molecules, where the sequence difference between the two input molecules corresponds to a difference between an amino acid sequence of each input molecule, and wherein the property difference between the two input molecules corresponds to a difference between a value of the property present in each in put molecule.

In another aspect, there is provided a computer-implemented method for training a property computation model. The method may include: identifying a set of sample molecules, where each sample molecule in the set of sample molecules is associated with a respective value for a property; generating a plurality of sample molecule pairs for inclusion in a training dataset, where the generating the plurality of sample molecule pairs includes pairing a first sample molecule and a second sample molecule selected from the set of sample molecules to generate a first sample molecule pair, and pairing the first sample molecule and a third sample molecule selected from the set of sample molecules to generate a second sample molecule pair; and training, based at least on the training dataset, a property computation model to determine, based at least on a sequence difference between two input molecules, a property difference between the two input molecules, where the sequence difference between the two input molecules corresponds to a difference between an amino acid sequence of each input molecule, and wherein the property difference between the two input molecules corresponds to a difference between a value of the property present in each in put molecule.

In another aspect, there is provided a computer program product including a non-transitory computer readable medium storing instructions. The instructions may cause operations may executed by at least one data processor. The operations may include: identifying a set of sample molecules, where each sample molecule in the set of sample molecules is associated with a respective value for a property; generating a plurality of sample molecule pairs for inclusion in a training dataset, where the generating the plurality of sample molecule pairs includes pairing a first sample molecule and a second sample molecule selected from the set of sample molecules to generate a first sample molecule pair, and pairing the first sample molecule and a third sample molecule selected from the set of sample molecules to generate a second sample molecule pair; and training, based at least on the training dataset, a property computation model to determine, based at least on a sequence difference between two input molecules, a property difference between the two input molecules, where the sequence difference between the two input molecules corresponds to a difference between an amino acid sequence of each input molecule, and wherein the property difference between the two input molecules corresponds to a difference between a value of the property present in each in put molecule.

In some variations, one or more features disclosed herein including the following features can optionally be included in any feasible combination.

In some variations, the generating the plurality of sample molecule pairs further includes pairing the second sample molecule with the third sample molecule to generate a third sample molecule pair.

In some variations, the property computation model includes a language model that has been trained to determine, based at least on the amino acid sequence of each input molecule, a relative embedding representative of the sequence difference between the two input molecules.

In some variations, the property computation model includes a neural network coupled with the language model, and the neural network has been trained to determine, based at least on the relative embedding, the property difference between the two input molecules.

In some variations, the property includes expression, affinity, specificity, developability, or biological activity.

In some variations, each sample molecule includes an antibody.

In some variations, each sample molecule includes a portion of an antibody.

In some variations, each sample molecule includes a variable region, an antigen binding region, a heavy chain, and/or a light chain of an antibody.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the design of large molecules such as protein molecules, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a molecule design system, in accordance with some example embodiments;

FIG. 2A depicts a flowchart illustrating an example of a process for machine learning based enhancement of molecular properties, in accordance with some example embodiments;

FIG. 2B depicts a flowchart illustrating an example of a process for generating a training dataset for training a property computation model, in accordance with some example embodiments;

FIG. 3 depicts a flowchart illustrating another example of a process for machine learning based enhancement of molecular properties, in accordance with some example embodiments;

FIG. 4A depicts a flowchart illustrating an example of a process for identifying mutations that cause an improvement in a property, in accordance with some example embodiments;

FIG. 4B depicts a flowchart illustrating an example of a process for identifying mutations that cause an improvement in a property, in accordance with some example embodiments;

FIG. 5A depicts a schematic diagram illustrating an example of a workflow for machine learning enabled enhancement of molecular properties, in accordance with some example embodiments;

FIG. 5B depicts a schematic diagram illustrating the sampling of point mutations across mutation sites and types of amino acid residues, in accordance with some example embodiments;

FIG. 6 depicts a schematic diagram illustrating a comparison of the relative embeddings of amino acid sequences that differ by a point mutation and by a higher edit distance, in accordance with some example embodiments

FIG. 7A depicts a graph illustrating the predicted and measured improvements in the binding affinity of variants of a lead molecule, in accordance with some example embodiments;

FIG. 7B depicts a graph illustrating the predicted and measured improvements in the binding affinity of variants of another lead molecule, in accordance with some example embodiments;

FIG. 7C depicts a graph illustrating the predicted and measured improvements in the binding affinity of variants of another lead molecule, in accordance with some example embodiments;

FIG. 7D depicts a graph illustrating the predicted and measured improvements in the binding affinity of variants of a lead molecule, in accordance with some example embodiments;

FIG. 7E depicts a graph illustrating the predicted and measured improvements in the binding affinity of variants of another lead molecule, in accordance with some example embodiments;

FIG. 7F depicts a graph illustrating the predicted and measured improvements in the binding affinity of variants of another lead molecule, in accordance with some example embodiments;

FIG. 8A depicts graphs illustrating affinity improvements as well as the expression and binding rates of molecules designed with mutations identified by leveraging a property computation model, in accordance with some example embodiments;

FIG. 8B depicts graphs illustrating affinity improvements as well as the expression and binding rates of molecules designed with mutations identified by leveraging a property computation model, in accordance with some example embodiments;

FIG. 8C depicts graphs illustrating affinity improvements as well as the expression and binding rates of molecules designed with mutations identified by leveraging a property computation model, in accordance with some example embodiments;

FIG. 8D depicts a sequence analysis of the highest-affinity molecules generated by modifying a lead molecule with mutations identified using a property computation model, in accordance withs some example embodiments;

FIG. 8E depicts a structural analysis of the highest-affinity molecules generated by modifying a lead molecule with mutations identified using a property computation model, in accordance withs some example embodiments;

FIG. 8F depicts a sequence analysis of the highest-affinity molecules generated by modifying another lead molecule with mutations identified using a property computation model, in accordance withs some example embodiments;

FIG. 8G depicts a structural analysis of the highest-affinity molecules generated by modifying another lead molecule with mutations identified using a property computation model, in accordance withs some example embodiments;

FIG. 9 depicts an evaluation of the performance of a protein computation model operating with different protein language models (pLMs) across three different lead molecules, in accordance with some example embodiments;

FIG. 10 depicts the effects of removing sample molecules with false annotations from the training dataset on the performance of a property computation model, in accordance with some example embodiments;

FIG. 11 depicts graphs illustrating that molecules modified with mutations identified using a property computation model maintained desirable properties even at higher edit distances, in accordance withs some example embodiments;

FIG. 12A depicts a surface plasmon resonance (SPR) sensorgram illustrating the binding interaction of the highest binding variant of a lead molecule modified with mutations identified using a property computation model, in accordance with some example embodiments;

FIG. 12B depicts a surface plasmon resonance (SPR) sensorgram illustrating the binding interaction of the highest binding variant of another lead molecule modified with mutations identified using a property computation model, in accordance with some example embodiments;

FIG. 12C depicts a surface plasmon resonance (SPR) sensorgram illustrating the binding interaction of the highest binding variant of another lead molecule modified with mutations identified using a property computation model, in accordance with some example embodiments; and

FIG. 13 depicts a block diagram illustrating an example of a computing system, in accordance with some example embodiments.

When practical, similar reference numbers denote similar structures, features, or elements.

### DETAILED DESCRIPTION

A molecule may be designed to exhibit multiple desirable properties including, in the case of therapeutics, drug-like properties such as binding affinity, specificity, biological activity, developability, and/or the like. Lead optimization is one variation of molecular design in which a lead molecule (or another select molecule) is modified to enhance desirable properties while minimizing undesirable ones. For example, a lead molecule identified as having clinically useful pharmacological or biological activities during early-stage development may have significant liabilities, such as chemical or physical instabilities, poor pharmacokinetics, immunogenicity, and/or the like. In instances where the lead molecule is a protein molecule, lead optimization may include modifying the underlying sequence of amino acid residues, for example, by changing the type of one or more constituent amino acid residues, such that the resulting candidate molecule exhibits better properties than the lead molecule. However, modifications that improve the lead molecule, particularly those that enhance desirable properties without introducing liabilities, may be difficult to identify at least because of the vast combinatorial space of possibilities. For instance, even for point mutations affecting a single amino acid residue, a lead molecule with an N quantity of amino acid residues can spawn 20N possible modifications. The number of possible modifications grows exponentially as the extent of the modifications in the lead molecule increases, for example, from a single to multiple amino acid residues. Due to limitations in time and laboratory resources, conventional lead optimization protocols relying on wet lab analysis (e.g., in vitro measurements, in vivo characterization, and/or the like) to examine the effects of each modification cannot adequately explore the possible modifications that can be made to a lead molecule.

While conventional, wet lab based approaches to molecular design are too resource intensive to support an expansive exploration of different molecule designs with an aim of identifying novel molecules with better properties than a known molecule, more recent efforts to leverage artificial intelligence and machine learning are challenged by the scarcity of labeled (or annotated) data. In fact, the procurement of data arising from the development of protein based therapeutics is also cost prohibitive due to the same limitations in time and laboratory resources. As such, computation models trained to minimize the need for wet lab analysis, such as those capable of predicting the properties of protein molecules, are often confined to low data regimes in which the quantity of training data is limited and the distribution of the training data fails to capture the full range of possible inputs that the computation models are likely to encounter in deployment. Computation models trained in low data regimes tend to overfit to the training data and perform poorly as a consequence. For example, if a training dataset contains too few protein molecules with known values for a particular property, a computation model trained on that training dataset may be unable to accurately predict the value of that property for protein molecules outside of distribution of the training dataset. Because a poor performing computation model cannot serve as an adequate in silico surrogate for wet lab analysis of modified lead molecules during lead optimization, the same bottlenecks still persist in the drug discovery process.

Various embodiments of the present disclosure improve the performance of computation models trained in low data regimes, particularly those deployed to predict the effects of modifications to the sequence of amino acid residues forming a protein molecule. For instance, in some example embodiments, a property computation model may be trained to determine the difference in the values of a property exhibited by pairs of different protein molecules instead of the absolute values of the property exhibited by individual protein molecules. In some cases, the property computation model may include a language model trained to generate a relative embedding representative of a difference in the sequences of amino acid residues forming the individual protein molecules in each pair of protein molecules. Moreover, in some cases, the property computation model may also include a neural network trained to determine, based at least on the relative embedding, the difference in the values of the property exhibited by the pair of protein molecules. As described in more details below, a small training dataset (e.g., ~200-1000 protein molecules with known values for the property) may be augmented combinatorically when training the property computation model to determine the difference in the values of the property exhibited by pairs of protein molecules. For instance, the combinatorial augmentation of the training dataset may increase the size of the training dataset as well as improve its distribution. Training on the combinatorically augmented training dataset may prevent overfitting and improve the out-of-distribution performance of the property computation model such that the property computation model can provide a faster and lower cost alternative to wet lab analysis.

In some example embodiments, the property computation model may be trained based on a training dataset in which each training sample includes a pair of sample protein molecules and a label indicating the difference between the known values of the property exhibited by each sample protein molecule. In some cases, the training dataset may be generated by combinatorically augmenting a set of sample protein molecules, each of which having a known value for the property, by pairing the same sample protein molecule with multiple other sample protein molecules. For example, in some cases, the training dataset may be generated to include a first training sample pairing a first sample protein molecule with a second sample protein molecule and a second training sample pairing the first sample protein molecule with a third sample protein molecule. In instances where the set of sample protein molecules includes N sample protein molecules, the aforementioned combinatorial augmentation may generate

$$( \begin{matrix}N \\ 2\end{matrix} )$$

pairs of sample protein molecules as training samples for inclusion in the training dataset. It should be appreciated that N sample protein molecules with known values for the property may not be a sufficient quantity for training a property computation model to accurately predict the absolute value of the property, especially for protein molecules that are outside of the distribution of the N sample protein molecules. Contrastingly, the

$$( \begin{matrix}N \\ 2\end{matrix} )$$

pairs of sample protein molecules, each having a known difference between the values of the property exhibited by each constitute sample protein molecule, may constitute a sufficiently large and well distributed training dataset for training the property computation model to accurately predict the difference in the values of the property exhibited by pairs of protein molecules. As described in more details below, the property computation model trained to predict the difference in the values of the property exhibited by pairs of protein molecules may be applied to explore possible modifications to a select molecule, such as a lead molecule, in a principled manner, thus improving the time and resource efficiency of molecular design.

In some example embodiments, the property computation model may be applied to determine how different mutations in the sequence of a lead molecule affect a property exhibited by the lead molecule, thereby identifying mutations that improve that particular property. In this context, an improvement may refer to an increase in the value of a desirable property or a decrease in the value of an undesirable property. In some cases, the property computation model may be applied to sample different mutations, including point mutations affecting a single amino acid residue in the sequence of the lead molecule and combinations of multiple point mutations. For example, in some cases, the property computation model may be applied to determine a difference in the values of a property exhibited by one or more pairs of input molecules, each of which including a first input molecule having a first amino acid sequence and a second input molecule having a second amino acid sequence. In some cases, at least one site in the first amino acid sequence may be occupied by a different type of amino acid residue than the same sites in the second input sequence. In the context of lead optimization, the first amino acid sequence may correspond to the sequence of the lead molecule having a first mutation while the second amino acid sequence may correspond to the sequence of the lead molecule having a second mutation. In instances where the output of the property computation model indicates that the first input molecule exhibits a better value for the property than the second input molecule, the first mutation that is present in the first amino acid sequence may be identified as improving the property. Accordingly, as described in more details below, a candidate molecule (e.g., for further assessment such as wet lab analysis) may be generated to include at least one mutation identified as improving the property.

In some example embodiments, the property computation model may be applied to sample different point mutations in order to identify one or more point mutations that cause an improvement to the property. In this context, a point mutation between two protein molecules may refer to a difference of a single amino acid residue in the corresponding amino acid sequences. Accordingly, in some cases, the property computation model may be applied to determine the difference in the values of the property exhibited by a plurality of pairs of input molecules, each of which including a first protein molecule having a first amino acid sequence and a second protein molecule having a second amino acid sequence. In some cases, the first amino acid sequence may remain fixed during the sampling process while the second amino acid sequence may be varied to exhibit a variety of different possible point mutation. For example, in some cases, the second amino acid sequence may be varied to exhibit, for one or more possible sites within the second amino acid sequence, a different type of amino acid residue than the type occupying the corresponding site in the first amino acid sequence. In some cases, the second amino acid sequence may be varied across all possible types of amino acid residues, such as the twenty canonical types of amino acid residues. As such, where a first type of amino acid residue occupies a particular site in the first amino acid sequence, the second type of amino acid residue occupying the same site in the second amino acid sequence may be varied in order to sample mutations across all other possible types of amino acid residues. In some cases, the variations across all possible types of amino acid residues may be repeated across every site within the second amino acid sequence. Doing so may constitute an exhaustive sampling of possible mutation sites and the different types of amino acid residues that can occupy each mutation site.

In some example embodiments, the property computation model may be applied to sample different combinations of point mutations in order to identify one or more combinations of point mutations that cause an improvement to the property. For example, in some cases, one or more combinations of point mutations may be generated from a set of point mutations identified as causing an improvement to the property. Each combination of point mutations may include two or more point mutations selected, for example, randomly, from the set of point mutations identified as causing an improvement to the property. The property computation model may then be applied to determine the difference in the values of the property exhibited by one or more pairs of protein molecules, each of which exhibiting a different combination of point mutations. In some cases, the property computation model may be applied in an iterative manner, for example, with a first protein molecule identified as having a better value for the property than a second protein molecule during one iteration, being paired with a third protein molecule exhibiting a different combination of point mutations than the second protein molecule in a next iteration. In instances where the first protein molecule is again determined to have a better value for the property than the third protein molecule, the first protein molecule may proceed to yet another iteration in which the first protein molecule is paired with a fourth protein molecule exhibiting a different combination of point mutations than the third protein molecule. In some cases, the combination of point mutations present in a protein molecule exhibiting a better value for the property than one or more other protein molecules may be identified as causing an improvement to the property.

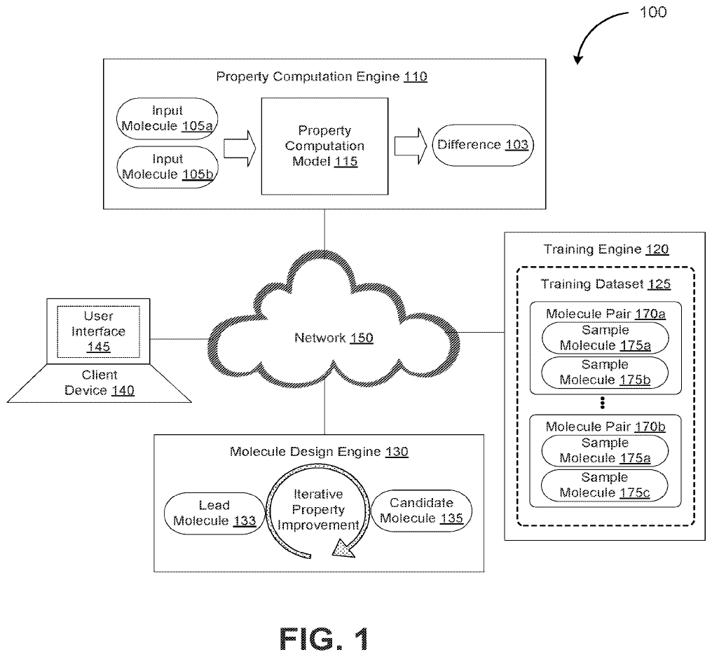

FIG. 1 depicts a system diagram illustrating an example of a molecule design system 100, in accordance with some example embodiments. Referring to FIG. 1 the molecule design system 110 may include a property computation engine 110, a training engine 120, a molecule design engine 130, and a client device 140. As shown in FIG. 1, the property computation engine 11, the training engine 120, the molecule design engine 130, and the client device 140 may be communicatively coupled via a network 150. The client device 140 may be a processor-based device including, for example, a workstation, a desktop computer, a laptop computer, a smartphone, a tablet computer, a wearable apparatus, and/or the like. The network 150 may be a wired network and/or a wireless network including, for example, a local area network (LAN), a virtual local area network (VLAN), a wide area network (WAN), a public land mobile network (PLMN), the Internet, and/or the like.

In some example embodiments, the property computation engine 110 may include a property computation model 115. In some cases, the property computation model 115 may have been trained, for example, by the property computation engine 120, to determine a difference 103 in the values of a property exhibited by a pair of molecules, such as the first input molecule 105a and the second input molecule 105b shown in FIG. 1. In some cases, the first input molecule 105a and the second input molecule 105b may be protein molecules for protein-based therapeutics including, for example, antibodies, chimeric antigen receptors (CARs), cytokines, hormones, enzymes, and/or the like. Accordingly, in some cases, the property of interest may be a drug-like property such as expression, affinity, specificity, immunogenicity, in vivo stability, self-association, and/or the like. As described in more details below, in some cases, the property computation model 115 may be applied to identify one or more mutations, including point mutations affecting a single amino acid residue and combinations of multiple point mutations that cause an improvement in the property. For example, in some cases, the molecule design engine 130 may apply the property computation model 115 to identify, over multiple iterations, one or more mutations that can be applied to a select molecule 133 in order to generate a candidate molecule 135 having a better value for the property than the select molecule 133. In some cases, the select molecule 133 may be a lead molecule that, in at least some molecular design applications, is a molecule identified as having clinically useful pharmacological or biological activities. The select molecule 133 may undergo iterative property improvement in order to enhance desirable properties while minimizing undesirable ones, thus reducing the likelihood of the resulting candidate molecule 135 exhibiting liabilities (e.g., chemical or physical instabilities, poor pharmacokinetics, immunogenicity, and/or the like) that can cause the candidate molecule 135 to fail as a viable therapeutic. Discovering such liabilities later in the drug development cycle can be especially catastrophic, which is why improving the properties of the select molecule 133 in the manner disclosed herein may reduce the long duration, high cost, and extraordinary risk associated with drug development.

Referring again to FIG. 1, the property computation model 115 may be trained based on a training dataset 125 generated by the training engine 120. In some cases, the training dataset 125 may include multiple pairs of sample molecules. Each pair of sample molecules may include a first sample molecule and a second sample molecule. Accordingly, in some cases, the property computation model 115 may be trained by at least applying the property computation model 115 to determine, for each pair of sample molecules in the training dataset 125, a property difference corresponding to a difference between a first value of a property exhibited by the first sample molecule and a second value of the property exhibited by the second sample molecule. Once trained, the property computation model 115 may be applied to determine, for each input molecule pair including a first input molecule and a second input molecule, the difference between a first value of the property exhibited by the first input molecule and a second value of the property exhibited by the second input molecule. As described in more details below, the trained property computation model 115 may be applied to identify one or more mutations, including point mutations affecting a single amino acid residue and combinations of point mutations that cause an improvement in the property. A select molecule, such as a lead molecule, may be modified to include these mutations such that the resulting candidate molecule (e.g., for wet lab analysis) exhibits better properties than the lead molecule.

In some example embodiments, the training engine 120 may generate the training dataset 125 by at least combinatorically augmenting a set of sample molecules, each of which having a known value for the property, by pairing the same sample molecule with multiple other sample molecules. For instance, in the example shown in FIG. 1, the training dataset 125 may be generated to include a first molecule pair 170a and a second molecule pair 170b. The first molecule pair 170a may be generated by pairing a first sample molecule 175a with a second sample molecule 175b. Furthermore, the first sample molecule 175a may also be paired with a third sample molecule 175b in order to form the second molecule pair 170b. Accordingly, where N sample protein molecules with known values for the property are available, the combinatorial expansion described herein may generate

$$( \begin{matrix}N \\ 2\end{matrix} )$$

molecule pairs for inclusion in the training dataset 175. While N sample molecules may be insufficient for training a property computation model to accurately predict the absolute value of the property exhibited by an input molecule,

$$( \begin{matrix}N \\ 2\end{matrix} )$$

molecule pairs may constitute a sufficiently large and well distributed training dataset for training the property computation model 115 to accurately predict the difference in the values of the property exhibited by one or more pairs of input molecules.

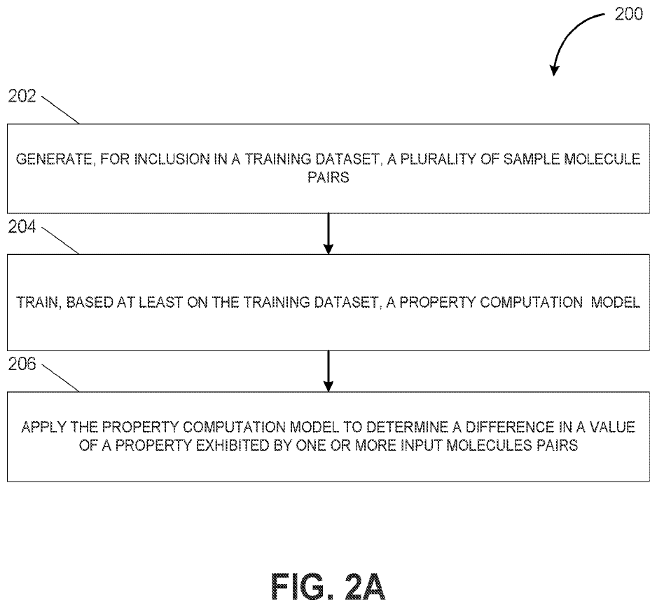

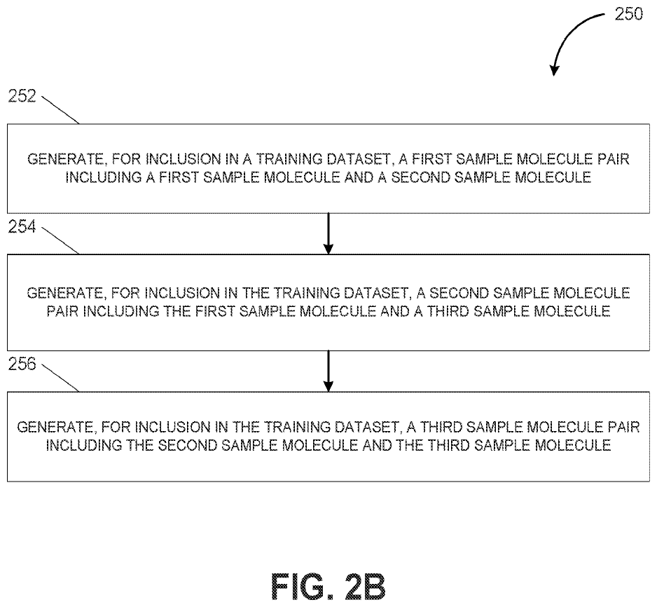

FIG. 2A depicts a flowchart illustrating an example of a process 200 for machine learning based enhancement of molecular properties, in accordance with some example embodiments. Referring to FIGS. 1 and 2A, in some example embodiments, the process 200 may be performed to train the property computation model 115 based on the training dataset 125.

At 202, a plurality of sample molecule pairs is generated for inclusion in a training dataset. In some example embodiments, a training dataset 125 may be generated to include multiple pairs of sample molecules. In some cases, each pair of sample molecules may include a first sample molecule having a first amino acid sequence and a second sample molecule having a second amino acid sequence. In some cases, the first amino acid sequence may exhibit at least one mutation relative to the second amino acid sequence. For example, in some cases, the at least one mutation may include two different types of amino acid residues occupying the same site location in the first amino acid sequence and the second amino acid sequence. As described in more details below, the training dataset may be generated by combinatorically augmenting a set of sample molecules, each of which being associated with a known value for the property. Combinatorial augmentation increases the volume of training data as well as improve its distribution. For instance, in some cases, N sample protein molecules may undergo combinatorial augmentation to generate

$$( \begin{matrix}N \\ 2\end{matrix} )$$

pairs of sample protein molecules for training the property computation model. Whereas the N sample protein molecules may be insufficient to train a property computation model to accurately predict the absolute value of the property exhibited by an input molecule, the

$$( \begin{matrix}N \\ 2\end{matrix} )$$

pairs of sample protein molecules may constitute a sufficiently large and well distributed training dataset for training the property computation model to accurately predict the difference in the values of the property exhibited by one or more pairs of input molecules. Further detail on generating a training dataset for training a property computation model is provided at exemplary FIG. 2B.

At 204, a property computation model is trained based at least on the training dataset. In some example embodiments, the property computation model 115 may be trained based on the training dataset 125 to determine, for each pair of sample molecules, a property difference corresponding to a difference between the first value of the property exhibited by the first sample molecule and the second value of the property exhibited by the second sample molecule. For example, in some cases, the property computation model may be trained to generate, for each pair of sample molecules, a relative embedding representative of a sequence difference corresponding to a difference between the first amino acid sequence of the first sample molecule and the second amino acid sequence of the second sample molecule. Furthermore, in some cases, the property computation model may be trained to determine, based at least on the relative embedding of each pair of sample molecules, the property difference corresponding to the difference between the first value of the property exhibited by the first sample molecule and the second value of the property exhibited by the second sample molecule. In some cases, the training of the property computation model may include adjusting one or more parameters (e.g., weights, biases, and/or of the like) of the property computation model to reduce (or minimize) an error in the output of the property computation model. For instance, in some cases, one or more parameters (e.g., weights, biases, and/or the like) of the property computation model may be adjusted in order to reduce (or minimize) the discrepancy (e.g., mean square error (MSE)) between the predicted difference in the value of the property determined by the property computation model and the known difference in the value of the property exhibited by each pair of sample molecules.

In some example embodiments, the property computation model may include a language model (e.g., a protein language model and/or the like) coupled with a neural network (e.g., a convolutional neural network and/or the like). For example, in some cases, the language model may be trained to generate a first embedding of the first amino acid sequence and a second embedding of the second amino acid sequence before determining a relative embedding corresponding to a difference between the first embedding and the second embedding. This paradigm for generating relative embeddings X_{i,j} for each pair of amino acid sequences {sᵢ, sⱼ} is shown as Equation (1) below, where the language model is denoted as F.

X_{i,j}=ΔEmb(sᵢ,sⱼ)=F_{θ}(sᵢ)−F_{θ}(sⱼ) (1)

In Equation (1), the language model is trained to generate the embeddings of each individual amino acid sequence sᵢ and sⱼ. Alternatively, it is also possible for the language model to be trained to generate the relative embedding directly, meaning that the language model may be trained to generate an output that approximates the difference between the first embedding of the first amino acid sequence and the second embedding of the second amino acid sequence. This alternative paradigm is illustrated in Equation (2).

X_{i,j}=F_{θ}(sᵢ,sⱼ) (2)

Accordingly, in some cases, the training of the property computation model may include training the language model to generate the relative embedding of each pair of sample molecules as well as training the neural network to determine, based at least on the relative embedding, the difference in the value of the property exhibited by each pair of sample molecules. In some cases, the training of the property computation model may include adjusting one or more parameters (e.g., weights, biases, and/or the like) of the language model such that the language model generates a relative embedding for each pair of sample molecules that enables the neural network to more accurately predict the difference between the constituent sample molecules. Furthermore, in some cases, the training of the property computation model may include adjusting one or more parameters (e.g., weights, biases, and/or the like) of the neural network to increase the accuracy of the predictions made by the neural network based upon the relative embeddings generated by the language model.

At 206, the property computation model is applied to determine a difference in a value of a property exhibited by one or more input molecule pairs. In some example embodiments, once trained, the property computation model 115 may be applied to determine the difference in the value of the property exhibited by one or more pairs of input molecules. For example, in some cases, the trained property computation model may be applied to determine the difference in the value of the property exhibited by a first input molecule and a second input molecule where a first amino acid sequence of the first input molecule exhibits one or more mutations relative to a second amino acid sequence of the second input molecule. As described in more detail below, applied in this manner, the property computation model may be leveraged to identify one or more mutations that cause an improvement in the property. For instance, in some cases, the property computation model may be applied to sample different point mutations, which affects a single amino acid residue, in order to identify those mutations that cause an improvement in the property. Alternatively and/or additionally, the property computation model may also be applied to sample different combinations of point mutations. One or more candidate molecules may be generated by at least modifying a selected molecule, such as a lead molecule, to include the one or more mutations identified as causing an improvement in the property.

FIG. 2B depicts a flowchart illustrating an example of a process 250 for generating a training dataset for training a property computation model, in accordance with some example embodiments. Referring to FIGS. 1 and 2A-2B, the process 250 may be performed, for example, by the training engine 120, to generate the training dataset 125. In some cases, the process 250 may implement operation 202 of the process 200 shown in FIG. 2A. As described in more details below, in some cases, the training dataset 125 may be generated through a combinatorial augmentation of a set of sample molecules with known values for the property. The combinatorial augmentation may include pairing each individual sample molecule with multiple other sample molecules in the set of sample molecules. In doing so, a training dataset including

$$( \begin{matrix}N \\ 2\end{matrix} )$$

pairs of sample molecules with known differences in the values of the property may be generated from a set of N sample protein molecules with known values for the property.

At 252, a first sample molecule pair including a first sample molecule and a second sample molecule is generated for inclusion in a training dataset. In some example embodiments, the training dataset for training a property computation model to determine the difference in the value of a property exhibited by one or more pairs of input molecules may be generated by combinatorically augmenting a set of sample molecules having known values for the property. In some cases, the combinatorial augmentation of the set of sample molecules may include pairing each sample molecule with multiple other sample molecules. For example, in some cases, a first pair of sample molecules may be generated for inclusion in the training dataset by at least pairing a first sample molecule with a second sample molecule. As described in more details below, the first sample molecule and/or the second sample molecule may be paired with other sample molecules in order to combinatorically augment the set of sample molecules.

At 254, a second sample molecule pair including the first sample molecule and a third sample molecule is generated for inclusion in the training dataset. In some example embodiments, a sample molecule that is already paired with one sample molecule to generate one pair of sample molecules in the training dataset may be paired with other sample molecules to generate additional pairs of sample molecules for inclusion in the training dataset. For example, the first sample molecule, which has been paired with the second sample molecule to generate the first pair of sample molecules, may be further paired with a third sample molecule in order to generate a second pair of sample molecules for inclusion in the training dataset.

At 256, a third sample molecule pair including the second sample molecule and the third sample molecule is generated for inclusion in the training dataset. In some example embodiments, the combinatorial augmentation of the set of sample molecules may be applied to every sample molecule available. For example, in some cases, the second sample molecule and the third sample molecule, which were paired with the first sample molecule to form the first and second pair of sample molecules, can also be paired to form a third pair of sample molecules for inclusion in the training dataset. Accordingly, where N sample protein molecules with known values for the property are available, the combinatorial expansion described herein may generate

$$( \begin{matrix}N \\ 2\end{matrix} )$$

molecule pairs for inclusion in the training dataset. As noted, in instances where the N sample protein molecules may be insufficient to train a property computation model to accurately predict the absolute value of the property exhibited by an input molecule, the

$$( \begin{matrix}N \\ 2\end{matrix} )$$

pairs of sample protein molecules may constitute a sufficiently large and well distributed training dataset for training the property computation model to accurately predict the difference in the values of the property exhibited by one or more pairs of input molecules.

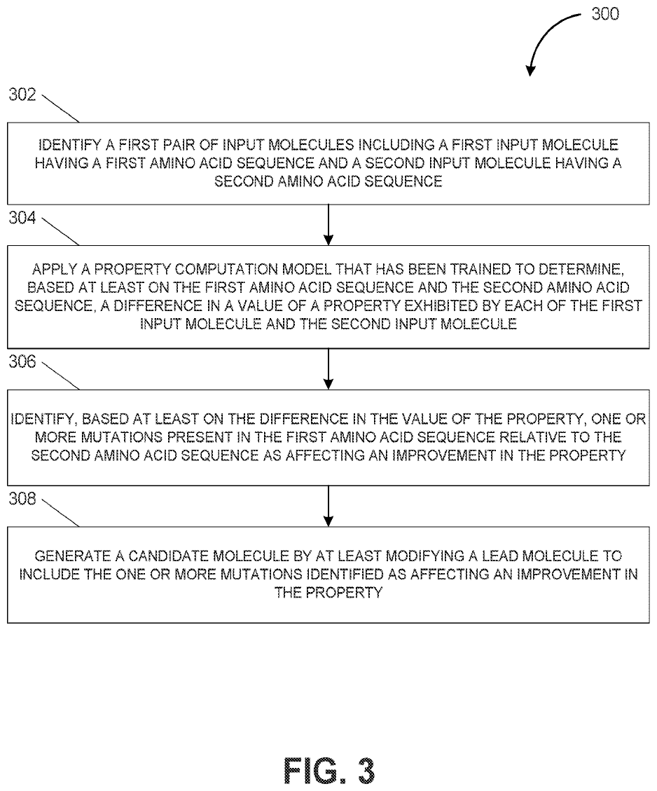

FIG. 3 depicts a flowchart illustrating another example of a process 300 for machine learning based enhancement of molecular properties, in accordance with some example embodiments. Referring to FIGS. 1 and 3, in some example embodiments, the property computation model 115 may be applied, for example, by the molecule design engine 130 in a workflow in which the lead molecule 133 is modified to generate the candidate molecule 135. For example, in some cases, the property computation model 115 may be applied to determine which one of two input molecules exhibits a better value for a drug-like property, such as expression, affinity, specificity, immunogenicity, in vivo stability, self-association, and/or the like. As described in more details below, in some cases, the property computation model 115 may be applied to identify one or more mutations, including point mutations affecting a single amino acid residue and/or combinations of multiple point mutations that cause an improvement in the property. Accordingly, in some cases, the candidate molecule 135 may be generated by at least modifying the lead molecule 133 to include one or more of the mutations identified by the property computation model 115 as causing an improvement in the property.

At 302, a first pair of input molecules including a first input molecule having a first amino acid sequence and a second input molecule having a second amino acid sequence is identified. In some example embodiments, a pair of input molecules including a first input molecule and a second input molecule may be generated such that a first amino acid sequence of the first input molecule exhibits one or more mutations relative to a second amino acid sequence of the second input molecule. In some cases, each mutation may be a point mutation in which two different types of amino acid residues occupy a same site in each of the first amino acid sequence and the second amino acid sequence. As described in more details below, a property computation model may be applied to evaluate the effects of different point mutations on the value of the property exhibited the first input molecule and the second input molecule. Alternatively and/or additionally, the property computation model may also be applied to evaluate the effects of different combinations of point mutations on the value of the property exhibited by the first input molecule and the second input molecule. Applying the property computation model in this manner may enable the identification of individual point mutations and, in some cases, combinations of point mutations that cause an improvement to the property.

At 304, a property computation model that has been trained to determine, based at least on the first amino acid sequence and the second amino acid sequence, a difference in a value of a property exhibited by each of the first input molecule and the second input molecule. In some example embodiments, a property computation model may be applied to determine, based at least on a relative embedding representative of the difference between the first amino acid sequence and the second amino acid sequence, a difference in the value of the property exhibited by each of the first input molecule and the second input molecule. For example, in some cases, the property computation model may include a language model (e.g., a protein language model (pLM) and/or the like) coupled with a neural network (e.g., a convolutional neural network (CNN) and/or the like). In some cases, the language model may have been trained to generate a first embedding of the first amino acid sequence and a second embedding of the second amino acid sequence. Moreover, the language model may have been further trained to determine, as the relative embedding, a difference between the first embedding and the second embedding. In some cases, the neural network may have been trained to determine, based at least on the relative embedding representative of the difference between the first amino acid sequence and the second amino acid sequence, the difference in the value of the property present in each of the first input molecule and the second input molecule.

At 306, one or more mutations present in the first amino acid sequence relative to the second amino acid sequence are identified as causing an improvement in the property based at least on the difference in the value of the property. In some example embodiments, the one or more mutations, whether a single point mutation or a combination of point mutation, that are present in the one of the first input molecule and the second input molecule having a better value for the property may be identified as causing an improvement to the property. For example, if the difference in the value of the property present in each of the first input molecule and the second input molecule indicates that the first input molecule exhibits a better value for the property, the one or more mutations that are present in the first amino acid sequence relative to the second amino acid sequence may be identified as causing an improvement to the property. In some cases, the one or more mutations may correspond to the types of amino acid residues occupying one or more sites in the first amino acid sequence that differ from the types of amino acid residues occupying the same sites in the second amino acid sequence.

At 308, a candidate molecule is generated by at least modifying a select molecule to include the one or more mutations identified as causing an improvement in the property. In some example embodiments, a candidate molecule with the one or more mutations identified as causing an improvement in the property may be generated for wet lab analysis (e.g., in vitro measurements, in vivo characterization, and/or the like). In some cases, generating the candidate molecule by modifying a select molecule, such as a lead molecule, to include the one or more mutations identified as causing the improvement in the property may enhance one or more desirable properties such as expression, affinity, specificity, in vivo stability, and/or the like. Alternatively and/or additionally, generating the candidate molecule by modifying a lead molecule to include the one or more mutations identified as causing the improvement in the property may also minimize one or more undesirable properties including liabilities such as chemical or physical instabilities, poor pharmacokinetics, immunogenicity, and/or the like.

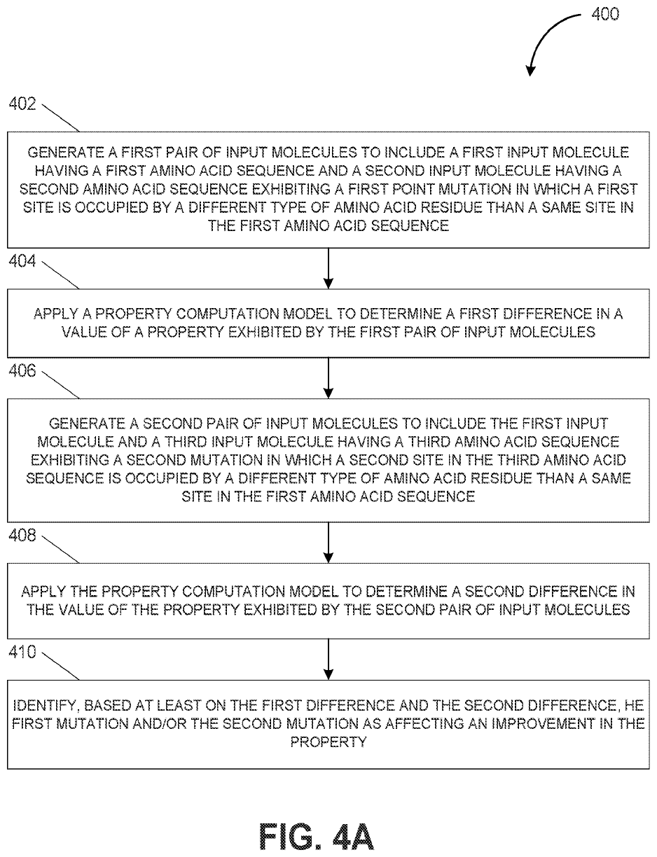

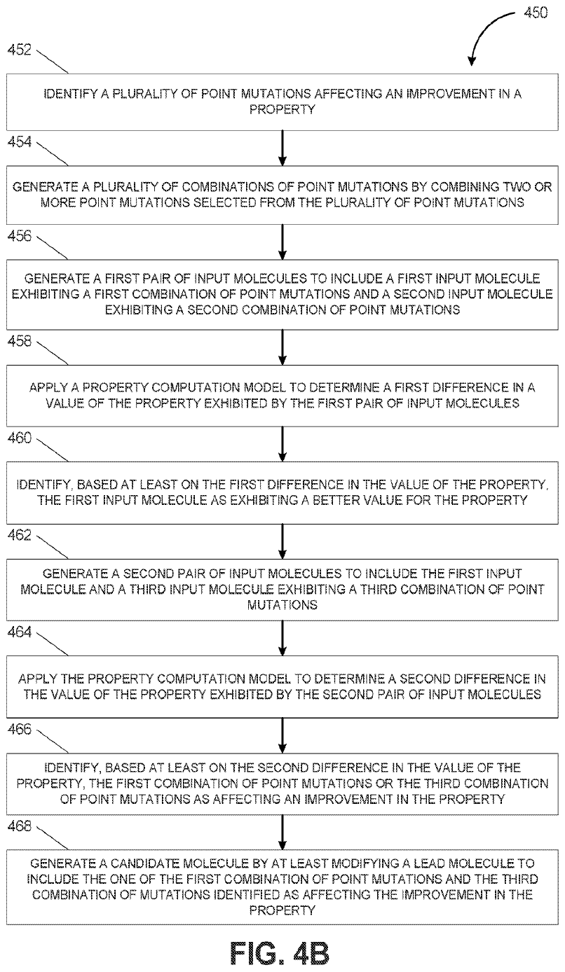

FIG. 4A depicts a flowchart illustrating an example of a process 400 for identifying mutations that cause an improvement in a property, in accordance with some example embodiments. Referring to FIGS. 1 and 4A, the process 400 may be performed, for example, by the molecule design engine 130, to identify one or more point mutations that cause an improvement in a property, such as expression, affinity, specificity, immunogenicity, in vivo stability, self-association, and/or the like. As noted, a point mutation affects a single amino acid residue, meaning that two input molecules may exhibit a point mutation if two different types of amino acid residues occupy a same site in each of the corresponding amino acid sequences. As described in more details below, one or more point mutations that cause an improvement in the property may be identified by sampling a variety of different point mutations. For example, in some cases, the sampling may be performed exhaustively across all possible mutation sites in input molecule and all types of amino acid residues (e.g., canonical amino acid residues) that can occupy each mutation site.

At 402, a first pair of input molecules is generated to include a first input molecule having a first amino acid sequence and a second input molecule having a second amino acid sequence exhibiting a first point mutation in which a first site is occupied by a different type of amino acid residue than a same site in the first amino acid sequence. In some example embodiments, a property computation model may be applied to sample different point mutations, including by evaluating the effects of having various different types of the amino acid residues at individual sites within a protein molecule. For example, in some cases, a first pair of input molecules may be generated to include a first input molecule having a first amino acid sequence that remains fixed during the sampling process. The first pair of input molecules may further include a second input molecule having a second amino acid sequence in which one site is occupied by a different type of amino acid residue than the same site in the first amino acid sequence. For instance, if a first site in the first amino acid sequence is occupied by leucine (L), the same first site in the second amino acid sequence may be occupied by alanine (A) instead. As described in more details below, that same first site may be further varied to be occupied by other types of amino acid residues including, in some cases, all possible other types of amino acid residues (e.g., canonical amino acid residues).

At 404, a property computation model is applied to determine a first difference in a value of a property exhibited by the first pair of input molecules. In some example embodiments, the effects of the first point mutation present in the second amino acid sequence may be evaluated by applying a property computation model to determine a difference in the value of the property exhibited by the first pair of input molecules. In some cases, the difference in the value of the property exhibited by the first pair of input molecules may indicate whether the first input molecule or the second input molecule exhibits a better value for the property. Moreover, in some cases, the first mutation present in the second amino acid sequence may be identified as causing an improvement in the property if the second input molecule is determined to exhibit a better value for the property than the first input molecule. In some cases, operations 402 and 404 may be repeated for every possible type of amino acid residue that can occupy the first site. For instance, in some cases, where the first site in the first amino acid sequence is occupied by leucine (L), the property computation model may be further applied to evaluate whether having tryptophan (W) occupy the same first site causes an improvement in the property or, in some cases, causes a greater improvement than alanine (A). As described in more details below, in addition to sampling across different possible types of amino acid residues for each individual site in the first amino acid sequence of the first input molecule, the sampling may further continue for one or more additional sites or, in some cases, every possible site, in an input molecule.

At 406, a second pair of input molecules is generated to include the first input molecule and a third input molecule having a third amino acid sequence exhibiting a second point mutation in which a second site is occupied by a different type of amino acid residue than a same site in the first amino acid sequence. In some example embodiments, to further sample the effects of having different types of amino acid residues at additional sites, a second pair of input molecules may be generated to include the first input molecule and a third input molecule in which a different type of amino acid residue occupies a second site in each of the amino acid sequences. For example, if the second site in the first amino acid sequence is occupied by glutamine (Q), the second mutation present in the third amino acid sequence may be the same second site being occupied by tyrosine (Y) instead.

At 408, a property computation model is applied to determine a second difference in the value of the property exhibited by the second pair of input molecules. In some example embodiments, the property computation model may be applied to determine whether the second mutation present in the third amino acid sequence causes an improvement in the property. For example, if the second site in the first amino acid sequence is occupied by glutamine (Q) while the same second site in the first amino acid sequence is occupied by tyrosine (Y), the difference in the value of the property exhibited by each of the first input molecule and the third input molecule may indicate whether the presence of glutamine (Q) or tyrosine (Y) in the second site is associated with a better value for the property. In some cases, the second mutation may be identified as causing an improvement in the property if the difference in the value of the property exhibited by the second pair of input molecules indicates that the third input molecule exhibits a better value for the property. Moreover, in some cases, operations 406 and 408 may be repeated for one or more additional sites or, in some cases, every possible site, in an input molecule.

At 410, the first mutation and/or the second mutation are identified, based at least on the first difference and the second difference, as causing an improvement in the property. In some example embodiments, a point mutation present in an amino acid sequence may be identified as causing an improvement in the property if the output of the property computation model indicates that the amino acid sequence exhibits a better value for the property than another amino acid sequence exhibiting a different point mutation in which, for example, a different type of amino acid residue occupies a same site. For example, when sampling the different types of amino acid residues that can occupy an individual site, point mutations that cause a threshold improvement in the property or, alternatively, a threshold quantity of point mutations that cause the greatest improvement in the property, may be identified. Moreover, in some cases, such point mutations may be identified for multiple sites or, in some cases, every possible site in an amino acid sequence. As described in more details below, a set of point mutations that cause an improvement in the property may be identified by combining two or more point mutations such that combinations of point mutations may be generated for further evaluation in which the property computation model is applied to identify one or more combinations of point mutations that cause an improvement in the property.

FIG. 4B depicts a flowchart illustrating an example of a process for identifying mutations that cause an improvement in a property, in accordance with some example embodiments. Referring to FIGS. 1 and 4B, the process 450 may be performed, for example, by the molecule design engine 130, to identify one or more combinations of point mutations that cause an improvement in a property, such as expression, affinity, specificity, immunogenicity, in vivo stability, self-association, and/or the like. In some cases, a single combination of point mutations may include two or more point mutations affecting two or more amino acid residues. As described in more details below, one or more combinations of point mutations may be generated by combining two or more point mutations identified, for example, by performing the process 400, as causing an improvement in the property. One or more pairs of input molecules, each of which including a first input molecule and a second input molecule exhibiting different combinations of point mutations, may be evaluated by applying a property computation model. One or more combinations of point mutations may be identified as causing an improvement in the property through an iterative process in which an input molecule having one combination of point mutations is identified by the property computation model as exhibiting a better value for the property during one iteration advances to a next iteration in which the property computation model is applied to determine a difference in the value of the property exhibited by that input molecule and another input molecule exhibiting a different combination of point mutations.

At 452, a plurality of point mutations that cause an improvement in a property are identified. In some example embodiments, a plurality of point mutations that cause an improvement in the property may be identified by sampling different point mutations, for example, by at least varying the types of amino acid residue that occupy one or more sites in an amino acid sequence. For example, in some cases, one or more iterations of the process 400 shown in FIG. 4A may be performed to evaluate the effects of having different types of amino acid residues in one or more sites in the amino acid sequence. In some cases, the plurality of point mutations, such as the presence of certain types of amino acid residues at one or more sites in the amino acid sequence, may be identified as causing an improvement to the property.

At 454, a plurality of combinations of point mutations are generated by combining two or more point mutations selected from the plurality of point mutations. In some example embodiments, multiple combinations of point mutations, each of which combining two or more point mutations identified as causing an improvement to the property, may be generated. It should be appreciated that while the point mutations may individually cause an improvement to the property, combining two or more such point mutations does not necessarily yield an improvement to the property. Accordingly, as described in more details below, combinations of two or more point mutations, each of which identified as causing an improvement to the property, may be evaluated in an iterative manner, for example, by applying a property computation model that has been trained to determine a difference in the value of the property exhibited by a pair of input molecules exhibiting different combinations of point mutations.

At 456, a first pair of input molecules are generated to include a first input molecule exhibiting a first combination of point mutations and a second input molecule exhibiting a second combination of point mutations. In some example embodiments, the first input molecule and the second input molecule may exhibit two different combinations of point mutations, with each point mutation having been identified as causing an improvement to the property, for example, by applying the property computation model to sample different point mutations. As described in more details, the property computation model may also be applied to identify combinations of point mutations that cause an improvement in the property. For example, in some cases, the property computation model may be applied in an iterative manner in which an input molecule identified as exhibiting a better value for the property during one iteration may advance to a next iteration where the property computation model is again applied to determine whether the same input molecule exhibits a better value for the property than another input molecule exhibiting a different combination of point mutations.

At 458, a property computation model is applied to determine a first difference in a value of the property exhibited by the first pair of input molecules. In some example embodiments, the property computation model may have been trained to determine the difference in the value of the property exhibited by two input molecules exhibiting two different combinations of point mutations. For example, in some cases, the property computation model may include a language model (e.g., a protein language model (pLM) and/or the like) that have been trained to generate a relative embedding representative of the difference between the amino acid sequence of each input molecule in the first pair of input molecules. Moreover, in some cases, the property computation model may include a neural network (e.g., a convolutional neural network (CNN) and/or the like) that has been trained to determine, based at least on the relative embedding, a difference in the value of the property exhibited by the first pair of input molecules.

At 460, the first input molecule is identified, based at least on the first difference in the value of the property, as exhibiting a better value for the property. In some example embodiments, the difference in the value of the property output by the property computation model may identify which one of the two input molecules exhibits a better value for the property. For example, the first input molecule may exhibit a better value for a desirable property, such as expression or affinity, if the first input molecule exhibits a higher value for that property than the second input molecule. Conversely, the first input molecule may exhibit a better value for an undesirable property, such as immunogenicity, if the first input molecule exhibits a lower value for that property than the second input molecule. As described in more detail below, the one input molecule exhibiting the better value for the property may advance to a next iteration in which it is evaluated against another molecule exhibiting a different combination of point mutations.

At 462, a second pair of input molecules are generated to include the first input molecule and a third input molecule exhibiting a third combination of point mutations. In some example embodiments, if the first input molecule in the first pair of input molecules is identified as exhibiting a better value for the property, that first input molecule may advance to a next iteration in which the first input molecule is paired with a third input molecule exhibiting a different combination of point mutations than the second input molecule. In some cases, the second combination of point mutations may also include individual point mutations that have been identified as causing an improvement in the property. As described in more details below, the property computation model may be applied to evaluate the second pair of input molecules.

At 464, the property computation model is applied to determine a second difference in a value of the property exhibited by the second pair of input molecules. In some example embodiments, the property computation model may be applied to determine, based at least on a relative embedding representative of a difference in the respective amino acid sequences of the first input molecule and the third input molecule, a difference in the value of the property exhibited by the second pair of input molecules. In some cases, the difference in the value of the property exhibited by the second pair of input molecules may identify one of the first input molecule and the third input molecule as exhibiting a better value for the property. In some cases, the iterative sampling of different combinations of point mutations may continue with either the first input molecule or the third input molecule advancing to another iteration in which it is paired with another input molecule exhibiting yet another different combination of point mutations. Furthermore, in some cases, it is also possible that the iterative sampling includes the property computation model being applied to identify which one of the second input molecule exhibiting the second combination of point mutations and the third input molecule exhibiting the third combination of point mutations has a better value for the property.

At 466, the first combination of point mutations or the third combination of point mutations are identified, based at least on the second difference in the value of the property, as causing an improvement in the property. In some example embodiments, one or more combinations of point mutations may be identified as causing an improvement in the property. For example, in some cases, the first combination of point mutations present in the first input molecule may be identified as causing an improvement in the property if the first input molecule exhibits a better value for the property than the third input molecule exhibiting the third combination of point mutations. It should be appreciated that the iterative sampling of different combinations of point mutations may continue until one or more criteria are met. For instance, in some cases, the one or more criteria may include having performed a threshold quantity of sampling iterations, each of which evaluating two different combinations of point mutations. Alternatively and/or additionally, the one or more criteria may include having identified a combination of mutations causing a threshold magnitude of improvement in the property.

At 468, a candidate molecule is generated by at least modifying a select molecule to include the one of the first combination of point mutations and the third combination of point mutations identified as causing the improvement in the property. In some example embodiments, a candidate molecule may be generated by modifying a lead molecule to include a combination of point mutations identified as causing an improvement in the property. In some cases, the candidate molecule including the combination of point mutations may synthesized for wet lab analysis, such as in vitro measurements, in vivo characterization, and/or the like. In some cases, generating the candidate molecule by modifying a lead molecule to include a combination of point mutations identified as causing the improvement in the property may enhance one or more desirable properties such as expression, affinity, specificity, in vivo stability, and/or the like. Alternatively and/or additionally, generating the candidate molecule by modifying a lead molecule to include the one or more mutations identified as causing the improvement in the property may also minimize one or more undesirable properties including liabilities such as chemical or physical instabilities, poor pharmacokinetics, immunogenicity, and/or the like. In some cases, modifying the lead molecule to include different combinations of two or more point mutations may further increase the diversity of the resulting candidate molecules, thus enabling the selection of a greater quantity of developable therapeutic candidates.

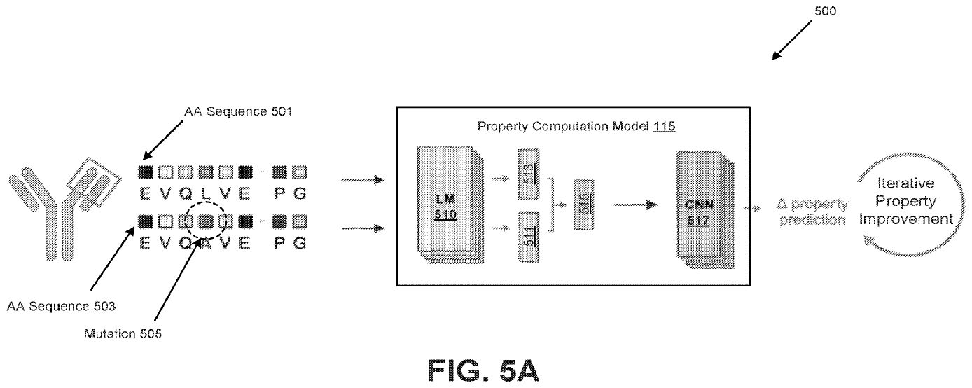

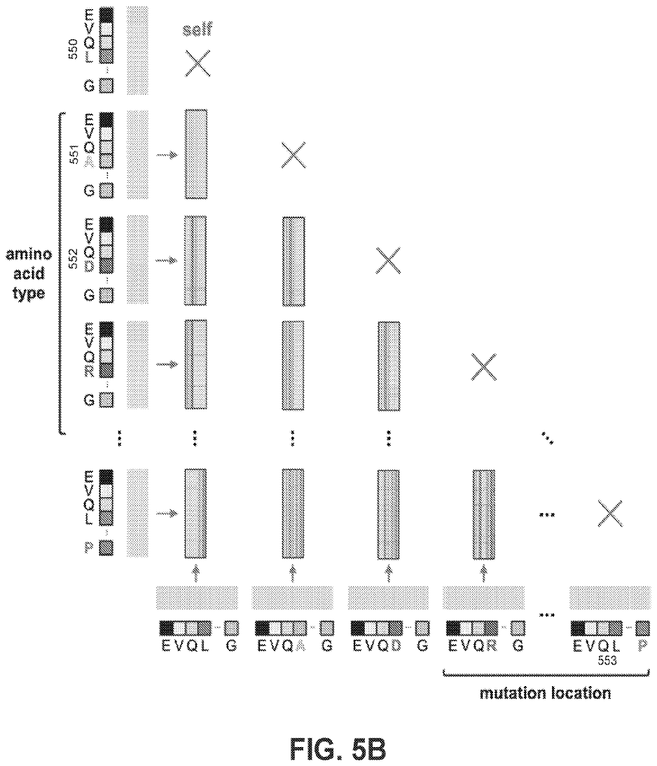

FIG. 5A depicts a schematic diagram illustrating an example of a workflow 500 for machine learning enabled enhancement of molecular properties, in accordance with some example embodiments. In the example of the workflow 500 shown in FIG. 5A, the property computation model 115 may be applied to determine a difference in the value of a property exhibited by each of a first amino acid sequence 501 and a second amino acid sequence 503. In some cases, each of the first amino acid sequence 501 and the second amino acid sequence 503 may be an antibody or, alternatively, a portion of an antibody such a paratope, variable region (Fv), antigen binding fragment (Fab), variable region, complementarity determining region (CDR), and/or the like. FIG. 5A depicts an example in which each of the first amino acid sequence 501 and the second amino acid sequence 503 is formed by concatenating variable domains on the heavy chain and light chain of an antibody.

Referring again to FIG. 5A, the first amino acid sequence 501 is different from the second amino acid sequence 503. In the example shown in FIG. 5A, the second amino acid sequence 503 exhibits a mutation 505 in which alanine (A) occupies a site in the second amino acid sequence 503 that is occupied by leucine (L) in the first amino acid sequence 501. The property computation model 115 may be applied to determine the difference in the value of the property exhibited by each of the first amino acid sequence 501 and the second amino acid sequence 503, thus determining whether the presence of alanine (A) instead of leucine (L) causes an improvement in the property. For example, FIG. 5A shows that the property computation model 115 includes a language model 510 (e.g., a protein language model (pLM) and/or the like) that has been trained to generate a first embedding 511 of the first amino acid sequence 501 and a second embedding 513 of the second amino acid sequence 503. In some cases, a relative embedding 515 representative of the difference between the first amino acid sequence 501 and the second amino acid sequence 503 may be generated. For instance, in some cases, the relative embedding 515 may be generated to corresponding to the difference between the first embedding 511 and the second embedding 513. As further shown in FIG. 5A, in some cases, a convolutional neural network 517 (or another type of neural network) may be applied to determine, based at least on the relative embedding 515, a difference in the value of the property (A property prediction in FIG. 5A).

In some example embodiments, the property computation model 115 may be applied to iteratively improve the value of the property. For example, the difference in the value of the property exhibited by each of the first amino acid sequence 501 and the second amino acid sequence 503 may indicate whether the mutation 505 (e.g., alanine (A) instead of leucine (L)) present in the second amino acid sequence 503 causes an improvement in the property. In instances where the second amino acid sequence 503 is determined to exhibit a better value for the property than the first amino acid sequence 501, the mutation 505 may be identified as causing an improvement in the property. In some cases, the effects of the mutation 505 may be further compared to one or more additional point mutations to identify a plurality of point mutations causing an improvement in the property. Moreover, in some cases, two or more point mutations causing an improvement in the property may be combined and the property computation model 115 applied to evaluate the effects of different combinations of point mutations. For instance, in some cases, one or more combinations of point mutations causing an improvement in the property may be identified through an iterative process in which an input molecule having one combination of point mutations identified as exhibiting a better value for the property during one iteration advances to a next iteration in which the property computation model is applied to determine a difference in the value of the property exhibited by that input molecule and another input molecule having a different combination of point mutations.

FIG. 5B depicts a schematic diagram illustrating the sampling of point mutations across mutation sites and types of amino acid residues, in accordance with some example embodiments. Referring to FIGS. 1, 4A, and 5B, in some example embodiments, the property computation model 115 may be applied to sample different point mutations such as in the process 400 shown in FIG. 4A. FIG. 5A further illustrates the sampling of different point mutations across types of amino acid residues and mutation sites. In the example shown in FIG. 5A, the property computation model 115 may be applied to determine a difference between the value of the property exhibited by an amino acid sequence 550 and variations of the amino acid sequence 550 with a type of amino acid residue occupying one or more individual sites. For example, in some cases, the property computation model 115 may be applied to determine the difference in the value of the property exhibited by the amino acid sequence 550 and a first variation of the amino acid sequence 551 in which alanine (A) occupies a particular site instead of leucine (L). Furthermore, the property computation model 115 may be applied to determine the difference in the value of the property exhibited by the amino acid sequence 550 and a second variation of the amino acid sequence 552 in which aspartic acid (D) occupies the site instead of leucine (L). The example in FIG. 5B further shows the sampling being performed for point mutations in at different mutation sites across the amino acid sequence 550. In some cases, the sampling may be exhaustive, meaning that the property computation model 115 is applied to determine the difference in the value of the property exhibited by the amino acid sequence 550 and variations of the amino acid sequence 550 across every mutation site and every possible type of amino acid residues (e.g., 20 canonical amino acid residues). In cases where a variation of the amino acid sequence 550 is determined to exhibit a better value for the property than the original amino acid sequence 550, the point mutation that is present in that variation of the amino acid sequence 550 (e.g., alanine (A) or aspartic acid (D) at a particular site instead of leucine (L)) may be identified as causing an improvement in the property. As noted, in some cases, two or more point mutations identified as causing an improvement in the property may be combined and the property computation model 115 may be applied to evaluate the effects of combinations of point mutations on the value of the property.

### Experimental Examples

In some example embodiments, the property computation model 115 was trained using labeled (or annotated) data from the Comprehensive Substitution for Multidimensional Optimization (COSMO) experiments, which mutationally scanned residues in antibody complementary-determining regions (CDRs) with all natural amino acids (except cysteine) to produce a dataset containing binding affinity labels for ~500-1000 point variants around a lead molecule. These ~500-1000 point variants and the corresponding binding affinity values (K_{d}) were combinatorically augmented to generate a training dataset with nearly 125,000 unique pairs of sample molecules. The property computation model 115 was trained on the ~125,000 pairs of sample molecules, each of which having a label (or annotation) corresponding to the difference in binding affinity exhibited by the constituent sample molecules.

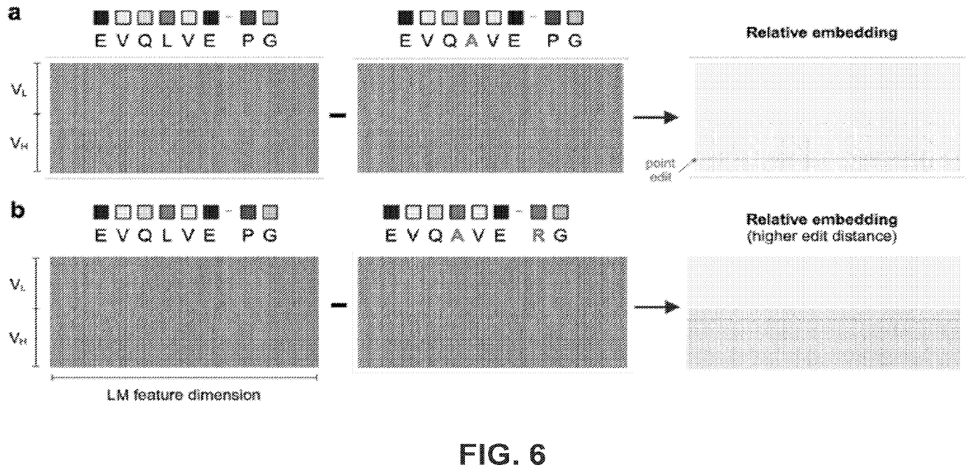

As noted, in some example embodiments, the property computation model 115 may operate on pairs of input molecules, such as the amino acid sequences of protein molecules. The amino acid sequences may be first fed through a pre-trained protein language model (pLM) that has been trained to extract embeddings of each individual amino acid sequence before determining a relative embedding representative of the differences therebetween. FIG. 6 illustrates the effects of different mutations on the relative embedding representative of the difference between two amino acid sequences. In the example shown in FIG. 6, each amino acid sequence is a concatenation of the variable region of the heavy chain (V_{H}) and the variable region of the light chain (V_{L}) of an antibody. Panel (a) shows the relative embedding of two amino acid sequences that differ by a single point mutation while Panel (b) shows the relative embedding of two amino acid sequences with a higher edit distance therebetween. While different types of protein language models may be used to implement the property computation model 115, it should be appreciated that in this context a protein language model (pLM) refers to a language model that has been trained exclusively or primarily on protein sequences (e.g., antibody sequences). This protein language model (pLM) is coupled with a convolutional neural net (CNN) trained on these relative embeddings and property labels to output a prediction of the property difference between two sequences. An example of this set up is shown in FIG. 5 where the language model 510 is coupled with the convolutional neural network 517. The property computation model 115 may be deployed as a ranking model to score combinations of known mutations or, as shown in FIG. 5A, as a part of a generative workflow to generate molecules with incrementally better property values.

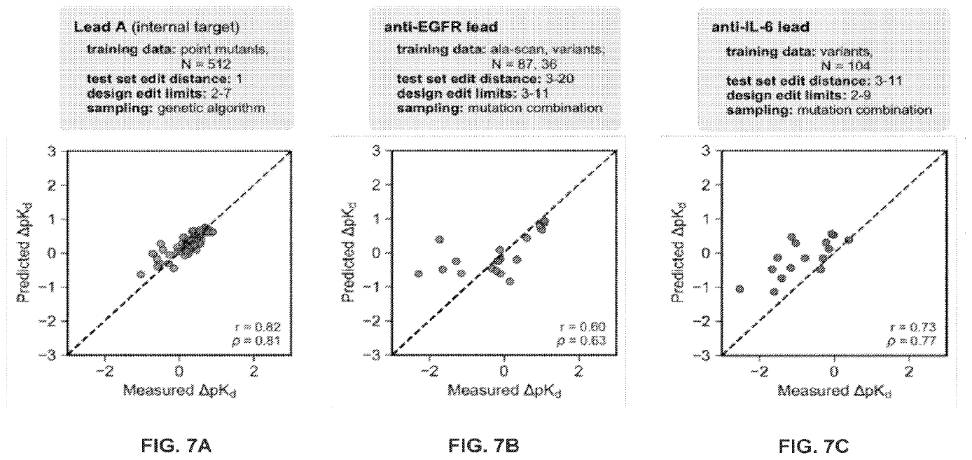

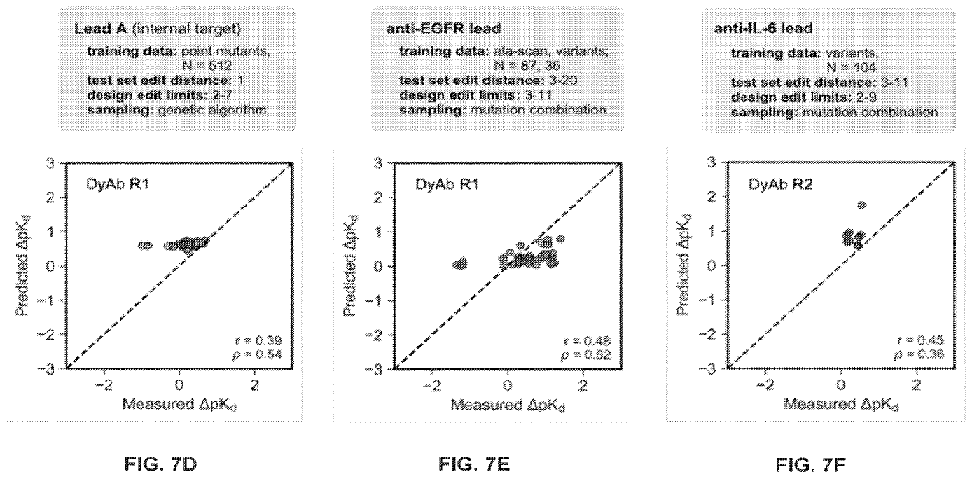

The performance of the property computation model 115 in determining the difference (e.g., improvement) in the value of a property, such as binding affinity, was evaluated on three antibody-antigen binding affinity datasets of varying size and complexity including lead molecule A, anti-EGFR, and anti-IL6. The results are shown in FIGS. 7A-C, which depicts graph illustrating the measured improvement in binding affinity and the improvement predicted by the property computation model 115. In this experiment, binding affinity is defined as the log transform of the equilibrium dissociation constant,

$$p {K}_{d} = - {log}_{10} {K}_{d} = ( \frac{{k}_{d}}{{k}_{a}} ) ,$$

wherein k_{d} and kₐ denote the measured dissociation and association rate constants, respectively. Each binding affinity dataset contains labels for variants around a lead molecule, which in this context is a known binder of a given antigen. Differences in binding affinity, denoted ΔpK_{d}, are then computed relative to the lead molecule.

Data preparation for training and testing the property computation model 115 included limited each binding affinity dataset to only those antibodies that bind the target antigen. As used herein, a “binder” is defined as having a measurable pK_{d} between 4 and 11. After a first round (R0) of designs against target A, noisy labels and those for which the surface plasmon resonance (SPR) curves are poorly fit (as assessed by visual inspection) are removed. The datasets for lead molecule A and anti-IL6 were randomly divided into an 85%/15% train/test split. The anti-EGFR training set contained all alanine-scanning point mutants and a randomly-selected half of the higher edit distance variants. The remaining half were held out for testing.

Antibody variable domain sequences were first aligned to the AHo-numbering scheme with ANARCI and concatenated to form variable domain sequences of consistent length (heavy followed by light chains). These sequences were then fed through and embeddings extracted from one of three pre-trained protein language models, with pLM A being a transformer-based masked language model trained on over 500 M natural antibody sequences from the Observed Antibody Space (OAS), pLM B being a 11-million parameter causal protein language model trained on antibodies in OAS as well as general proteins from UniRef50 and protein complexes in the Protein Databank (PDB33), and pLM C being a general protein language model trained on ~65 M protein sequences from the UniRef5032 dataset.

Embeddings generated by the protein language model were frozen, resized, normalized by the minimum tensor value, and rescaled based on the tensor maximum. Relative embeddings X_{i,j} for each pair of amino acid sequences {sᵢ, sⱼ} were obtained by element-wise subtraction of the normalized embeddings in accordance with Equation (3) below.

X_{i,j}=ΔEmb(sᵢ,sⱼ)=F_{θ}(sᵢ)−F_{θ}(sⱼ) (3)

wherein F_{θ} denotes the protein language model (pLM). That is, in some cases, the protein language model (pLM) may be trained to generate individual embeddings while the relative embedding is the difference between the individual embeddings. Alternatively, as noted, it is also possible for the relative embeddings X_{i,j} to be generated by the protein language model (pLM) directly approximating the difference between the individual embeddings. In this case, the protein language model (pLM) does not generate embeddings for each individual amino acid sequence but produces an output that approximates the difference between the embeddings of the individual amino acid sequences. For the downstream task, these relative embeddings X_{i,j} were used as input for a convolutional neural network (CNN) that has been trained to predict the property difference between the amino acid sequences sᵢ and sⱼ. A ResNet18 architecture as implemented in PyTorch and an Adam optimizer with a learning rate of 2E-4 was employed. Models were trained for 100 epochs using a mean-square error loss function and a batch size of 32. For inference, the relative embedding to the lead molecule X_{i,lead} was computed for a given protein language model Fe, and used to predict the property difference relative to the lead molecule.

Given the foregoing framework, the property computation model 115 performed well on the held-out test set N=77 point variants of the first dataset, which is associated with lead molecule A, achieving a Pearson correlation coefficient of r=0.82 and Spearman rank correlation coefficient of ρ=0.81 (see FIG. 7A). The other two binding affinity datasets for anti-EGFR and anti-IL6 lead molecules, with only ~100 affinity labels for each. These binding affinity datasets also included more distant variants of the lead molecule, within an edit distance of 20. Such variants are typically be obtained via multiple rounds of in silico and/or in vitro affinity maturation. For the anti-EGFR lead molecule where the property computation model 115 was trained on point-variant data from alanine-scanning mutagenesis and a handful of higher edit distance variants, the property computation model 115 was able to predict the difference in binding affinity ΔpK_{d} of the variants with a Pearson correlation coefficient of r=0.60 and a Spearman rank correlation coefficient of p=0.63 (see FIG. 7B). The property computation model 115 performed well even in the complete absence of point-variant data, as is the case with the anti-IL6 lead molecule shown in FIG. 7C, with a Pearson correlation coefficient of r=0.73 and a Spearman rank correlation coefficient of p=0.77.

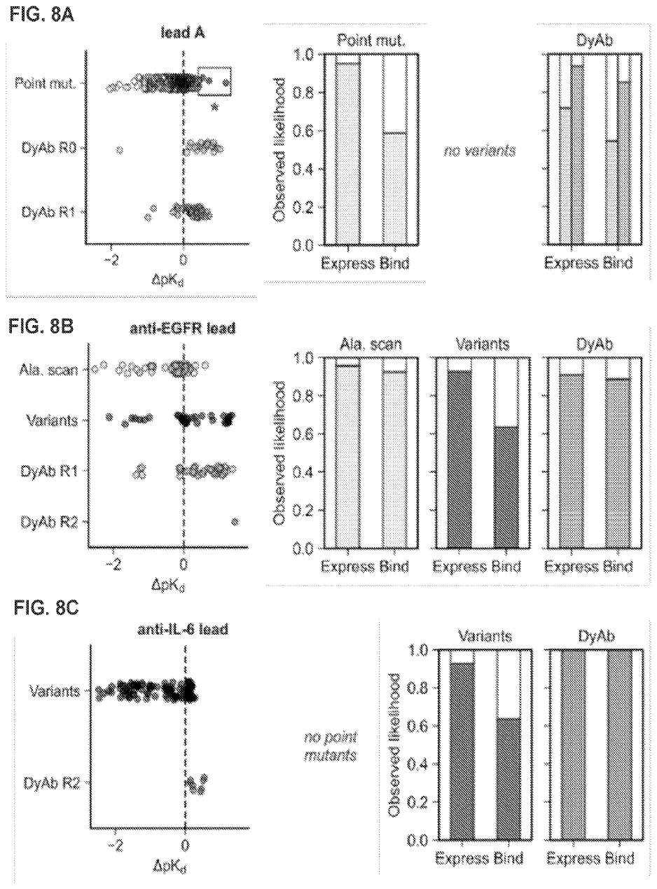

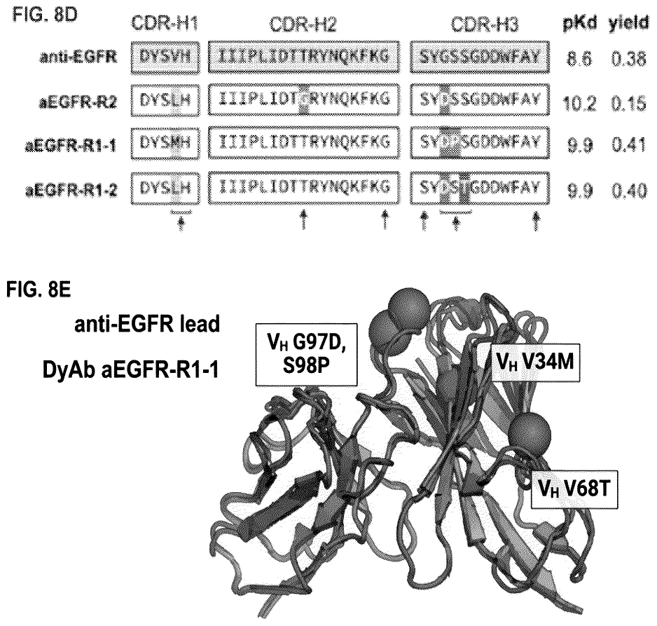

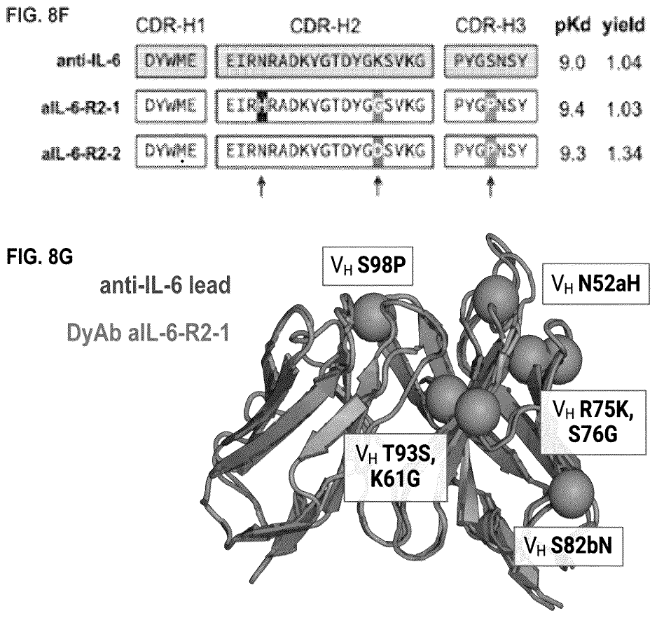

The performance of the property computation model 115 when leveraged to generate novel variants of a lead molecule with better binding affinity was also evaluated. For the lead molecule A set, all COSMO mutations that individually improved affinity were selected and used to generate combinations with an edit distance of 3 to 4. The property computation model 115 was then to determine the difference in binding affinity, or ΔpK_{d}, of variants exhibiting these combinations of COSMO mutations relative to the lead molecule. The generation of novel variants of the lead molecule was performed iteratively, with a genetic algorithm (GA) being employed to select and mutate the amino acid sequences, sample the vast design space, and iteratively improve the predicted ΔpK_{d}. The 46 top-ranked designs produced based on the output of the property computation model 115 were selected for experimental testing, with the results shown in FIG. 8A. As shown in FIG. 8A, 72% of the designs in this first round (R0) successfully expressed in mammalian cells while 54% bound to the target antigen, a comparable binding rate to that of the COSMO point mutants (59%). The performance of the property computation model 115 on the regression task for design sets are shown in FIGS. 7D-7F.

A similar procedure was followed to design variants of the anti-EGFR lead molecule. First, all point mutations in the training set that improved affinity individually (in the alanine-scan) or those within a higher edit distance variant were selected. Instead of employing a genetic algorithm, all combinations of these mutations between edit distances of 3 and 11 were sampled exhaustively and scored with the property computation model 115. Any design with a better binding affinity than the lead molecule (e.g., ΔpK_{d}>0) were further narrowed to 44 final amino acid sequences for experimental testing. In the first round (“R1”), 91% and 89% of designs expressed and bound to EGFR, respectively (See FIG. 8B, R1), with 79% of these binders exhibiting stronger measured affinities than that of the lead molecule (3.0 nM). Eleven designs showed at least a ten-fold improvement in binding affinity. While the strongest binders had similar affinities to high edit distance variants in the training set (~100 pM), leveraging the property computation model 115 produced binders at a much higher rate, on par with that of alanine point mutants. A second round of design (“R2”) that incorporated these data back into the training set was then performed. A single sequence from the second design round (“R2”) was selected for experimental testing (FIG. 8B, R2), and this design expressed, bound EGFR, and exhibited an improved affinity of 63 pM.

The performance of the property computation model 115 was also tested when the given dataset ~100 variants of the lead molecule, as is the case with the anti-IL6 lead molecule. Similarly, all mutations from affinity-improving variants with an edit distance of up to 9 were selected and exhaustively combined. The resulting amino acid sequences were selected and scored with the property computation model 115. A subset of 8 designs that the output of the property computation model 115 indicated as having a greater binding affinity than the lead molecule (e.g., ΔpK_{d}>0) were selected for experimental testing (see FIG. 8C, R2). All of the selected variants successfully expressed, bound to IL6, and exhibited better binding affinity than the lead molecule (1.4 nM). Four designs exhibited a better than 3-fold improvement in binding affinity than the lead molecule, surpassing all variants in the training set.

While the property computation model 115 is a sequence-based model, analysis of top designs suggests putative structural mechanisms underlying the observed improvement in biding affinity. The variable domain (Fab) structures of the anti-EGFR lead molecule and that of the top first round variants designed leveraging the property computation model 115 were determined experimentally via x-ray crystallography to resolutions of 2.4 angstroms (PDB entry) and 2.1 angstroms (PDB entry), respectively. The variable domains are overlaid in FIG. 8F. The output of the property computation model 115 identified in aEGFR-R1-1 across CDR-H1, CDR-H3, and the framework region. Mutating Kabat position VH 97 from glycine to aspartic acid—consistent across all top designs (FIG. 8E)—alters the conformation of CDR-H3. An additional proline at VH 98 could further stabilize this binding-able conformation in aEGFR-R1-1 relative to the lead molecule. All three top designs also mutate the VH 34 at the base of CDR-H1 from valine to a longer aliphatic amino acid. The highest affinity design (aEGFR-R2) includes 9 total edits, including 6 in the framework. These distal mutations could negatively impact expression as the yield for aEGFR-R2 was less than half that of the lead molecule (FIG. 8E).

In lieu of experimental structures, the structures of highest binding affinity anti-IL6 variants generated using the property computation model 115 were predicted computationally using ABodyBuilder219 (see FIG. 8H). While the same VH S98P mutation is seen in the top two designs, the predicted CDR-H3 conformation remains unchanged. Instead, a mutation in CDR-H2 (VH N52aH) drives extension of the loop into solution (see FIG. 8G).

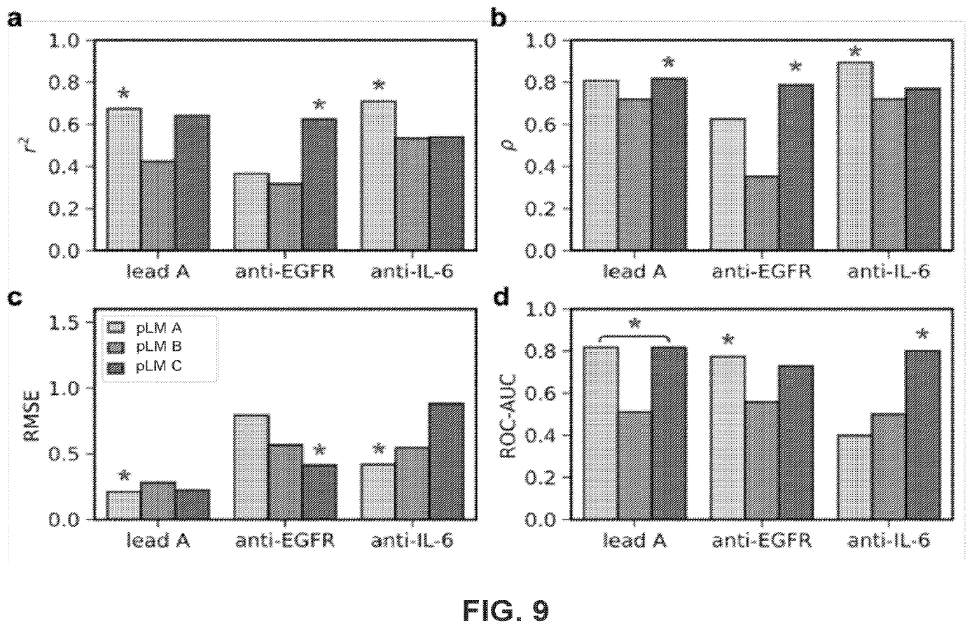

The pair-wise framework of property computation model 115 can be constructed from a variety of language model embeddings, including domain specific protein language models (e.g., antibody-specific pLMs) and more general protein language models. FIG. 9 compares the performance of three different variations of the property computation model 115, each of which including a different type of protein language model. In this example, pLM A is an antibody-specific protein language model that was trained on 500 million natural antibody sequences, pLM B is a hybrid that was trained on the same antibody sequences plus the general protein universe, and pLM C is a generic protein language model that was trained on just the general protein universe. The performance of the property language model 115 with different protein language models is compared across all three binding affinity datasets, lead molecule A, anti-EGFR, and anti-IL6.

FIG. 9 shows that performance on the downstream ΔpK_{d} prediction task varies between datasets. In some cases, better performance with a different protein language model than was used for design. For example, the anti-IL6 R2 designs were generated using embeddings from pLM B, which are outperformed by pLM A across three of the four evaluation metrics. However, protein language models trained primarily or entirely on antibody repertoires (e.g., pLM A and B) produced the most predictive variations of the property computation model 115 across the board compared to a general protein language model (e.g., pLM C).

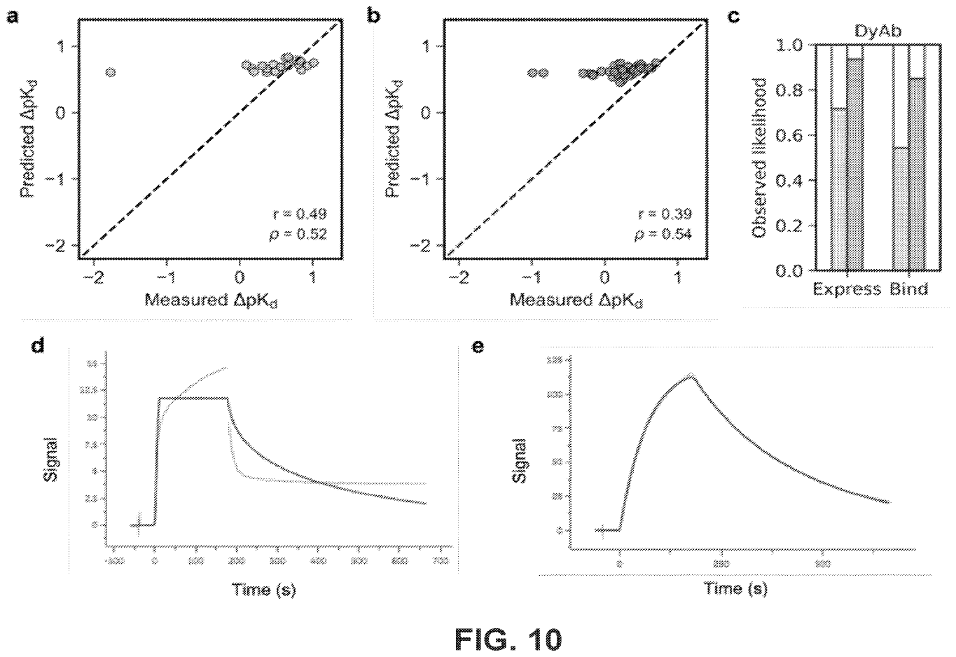

FIG. 10 shows that the rate of generating binders while leveraging the output of the property computation model 115 predicting the difference in binding affinity ΔpK_{d} can be further improved through data cleaning. For example, FIGS. 10(a) and (b) show that the differences in binding affinity ΔpK_{d} predicted by the property computation model are comparable after data cleaning between the first design round R0 (FIG. 10(a)) and the second design round R1 (FIG. 10(b)). Removing five falsely labeled binders from the training dataset improves both the expression (FIG. 10(c)) and binding rate (FIG. 10(d)) of designs generated using the property computation model 115. Example surface plasmon resonance (SPR) sensorgrams are shown for incorrectly (FIG. 10(d)) and correctly labeled (FIG. 10(e)) datapoints.

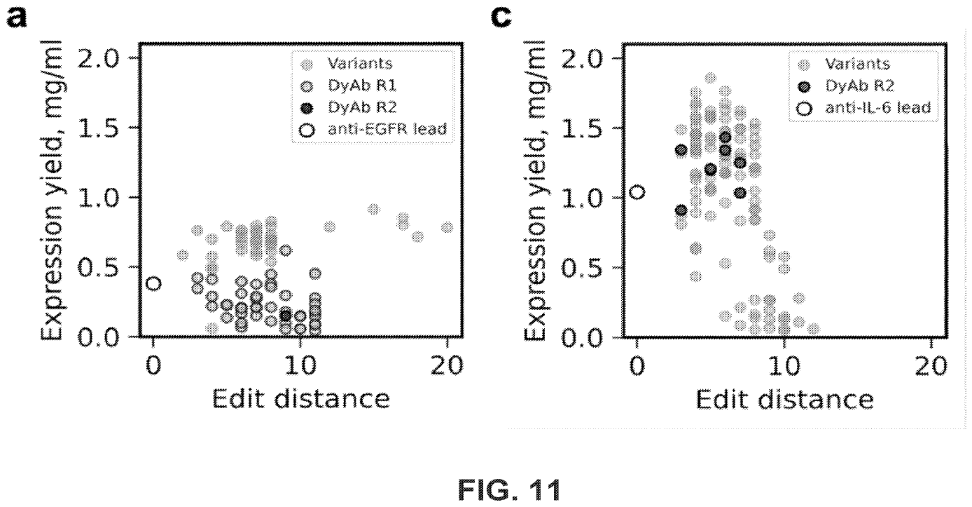

FIG. 11 shows that the designs generated using the property computation model 115 maintain affinity and expression at higher edit distances. Expression levels versus edit distance for variants in the training set and designs generated using the property computation model 115 are shown for the anti-EGFR lead molecule (FIG. 11(a)) and the anti-IL6 lead molecule (FIG. 11(b)).

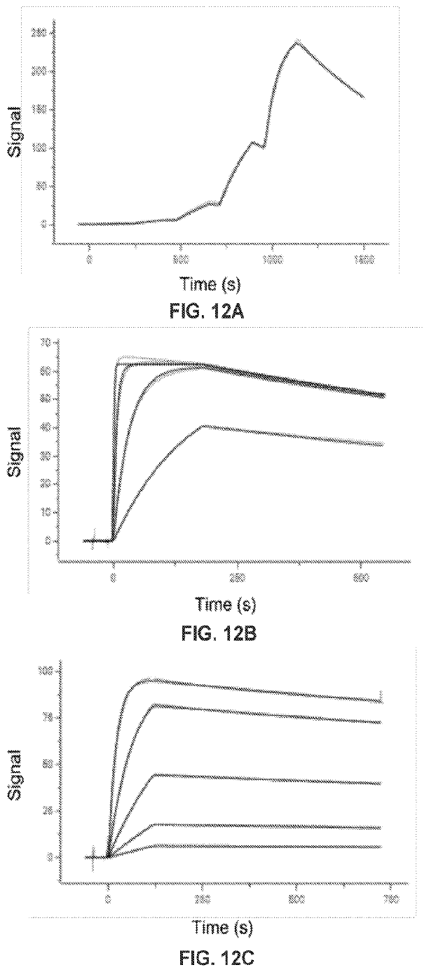

FIG. 12 depicts the raw surface plasmon resonance (SPR) curves for the top designs generated using the property computation model 115 for lead molecule A (FIG. 12(a)), the anti-EGFR lead molecule (FIG. 12(b)) and the anti-IL6 lead molecule (FIG. 12(c)). The measurement mode was changed from single-cycle to multi-cycle kinetics in between testing for lead molecule A and the anti-EGFR lead molecule.

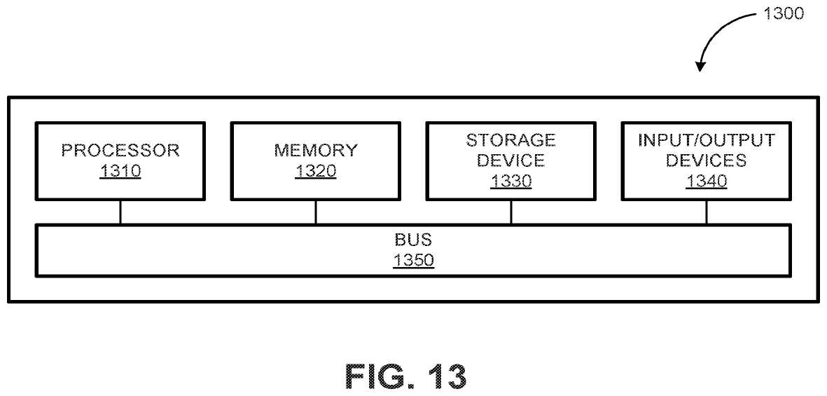

FIG. 13 depicts a block diagram illustrating an example of a computing system 1300, in accordance with some example embodiments. Referring to FIGS. 1-13, the computing system 1300 may be used to implement the property computation engine 110, the training engine 120, the molecule design engine 110, the client device 140, and/or any components therein.

As shown in FIG. 13, the computing system 1300 can include a processor 1310, a memory 1320, a storage device 1330, and input/output devices 1340. The processor 1310, the memory 1320, the storage device 1330, and the input/output devices 1340 can be interconnected via a system bus 1350. The processor 1310 is capable of processing instructions for execution within the computing system 1300. Such executed instructions can implement one or more components of, for example, the property computation engine 110, the training engine 120, the molecule design engine 110, the client device 140, and/or the like. In some example embodiments, the processor 1310 can be a single-threaded processor. Alternately, the processor 1310 can be a multi-threaded processor. The processor 1310 is capable of processing instructions stored in the memory 1320 and/or on the storage device 1330 to display graphical information for a user interface provided via the input/output device 1340.

The memory 1320 is a computer readable medium such as volatile or non-volatile that stores information within the computing system 1300. The memory 1320 can store data structures representing configuration object databases, for example. The storage device 1330 is capable of providing persistent storage for the computing system 1300. The storage device 1330 can be a floppy disk device, a hard disk device, an optical disk device, or a tape device, or other suitable persistent storage means. The input/output device 1340 provides input/output operations for the computing system 1300. In some example embodiments, the input/output device 1340 includes a keyboard and/or pointing device. In various implementations, the input/output device 1340 includes a display unit for displaying graphical user interfaces.

According to some example embodiments, the input/output device 1340 can provide input/output operations for a network device. For example, the input/output device 1340 can include Ethernet ports or other networking ports to communicate with one or more wired and/or wireless networks (e.g., a local area network (LAN), a wide area network (WAN), the Internet).

In some example embodiments, the computing system 1300 can be used to execute various interactive computer software applications that can be used for organization, analysis and/or storage of data in various formats. Alternatively, the computing system 1300 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1340. The user interface can be generated and presented to a user by the computing system 1300 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor; and
at least one memory storing instructions, which when executed by the at least one data processor, result in operations comprising:
generating a training dataset including a pair of sample molecules, the sample molecule pair including a first molecule and a second molecule;
training, based at least on the training dataset, a property computation model, wherein the property computation model is trained to at least,
determine a sequence difference between the first molecule and second molecule, the sequence difference comprising a difference between an amino acid sequence of the first molecule and an amino acid sequence of the second molecule,
generate a relative embedding representative of the sequence difference and associating the sequence difference with the sample molecule pair, and
determine, based at least on the relative embedding of the sample molecule pair, a property difference for the sample molecule pair, where the property difference for the molecule pair corresponds to a difference between a value of the property present in the first molecule and a value of the property present in the second molecule; and
applying the property computation model to determine a property difference between a pair of input molecules.

2. The system of claim 1, wherein the property comprises expression, affinity, specificity, developability, or biological activity.

3. The system of claim 1, wherein the sample molecule pair is generated by at least pairing sample molecules from a set of sample molecules having known values for the property.

4. The system of claim 1, wherein the generating the training dataset includes
pairing a first sample molecule and a second sample molecule from a set of sample molecules to generate the sample molecule pair, and
pairing the first sample molecule with a third sample molecule from the set of sample molecules to generate another sample molecule pair.

5. The system of claim 1, wherein the property computation model includes a language model, and wherein the training the property computation model includes training the language model to generate the relative embedding of the sample molecule pair.

6. The system of claim 5, wherein the language model is trained to at least
generate, for the sample molecule pair, a first embedding for the first sample molecule and a second embedding for the second sample molecule, and
generate the relative embedding of each sample molecule pair by at least determining a difference between the first embedding and the second embedding.

7. The system of claim 6, wherein the difference between the first embedding and the second embedding corresponds to the sequence difference comprising the difference the amino acid sequence of the first molecule and the amino acid sequence of the second molecule.

8. The system of claim 5, wherein the property computation model further includes a neural network coupled with the language model, and wherein the training the property computation model includes training the neural network to determine, based at least on the relative embedding of each sample molecule pair, the property difference for each sample molecule pair.

9. The system of claim 1, further comprising:
identifying, based at least on the property difference of the pair of input molecules, one or more mutations associated with an improvement to the property.

10. The system of claim 9, wherein the one or more mutations include a point mutation where the pair of input molecules differ by a single position in each corresponding amino acid sequence.

11. The system of claim 9, wherein the one or more mutations include a combination of multiple point mutations where the pair of input molecules differ by multiple positions in each corresponding amino acid sequence.

12. The system of claim 1, wherein each sample molecule comprises an antibody or a portion of the antibody.

13. The system of claim 1, wherein each sample molecule comprises a variable region, an antigen binding region, a heavy chain, and/or a light chain of an antibody.

14. A computer-implemented method, comprising:
generating a training dataset including a pair of sample molecules, the sample molecule pair including a first molecule and a second molecule;
training, based at least on the training dataset, a property computation model, wherein the property computation model is trained to at least,
determine a sequence difference between the first molecule and second molecule, the sequence difference comprising a difference between an amino acid sequence of the first molecule and an amino acid sequence of the second molecule,
generate a relative embedding representative of the sequence difference and associating the sequence difference with the sample molecule pair, and
determine, based at least on the relative embedding of the sample molecule pair, a property difference for the sample molecule pair, where the property difference for the molecule pair corresponds to a difference between a value of the property present in the first molecule and a value of the property present in the second molecule; and
applying the property computation model to determine a property difference between a pair of input molecules.

15. A non-transitory computer readable medium storing instructions, which when executed by the at least one data processor, result in operations comprising:
generating a training dataset including a pair of sample molecules, the sample molecule pair including a first molecule and a second molecule;
training, based at least on the training dataset, a property computation model, wherein the property computation model is trained to at least,
determine a sequence difference between the first molecule and second molecule, the sequence difference comprising a difference between an amino acid sequence of the first molecule and an amino acid sequence of the second molecule,
generate a relative embedding representative of the sequence difference and associating the sequence difference with the sample molecule pair, and
determine, based at least on the relative embedding of the sample molecule pair, a property difference for the sample molecule pair, where the property difference for the molecule pair corresponds to a difference between a value of the property present in the first molecule and a value of the property present in the second molecule; and
applying the property computation model to determine a property difference between a pair of input molecules.

16-55. (canceled)

56. The system of claim 9, wherein the identifying the one or more mutations includes
identifying a mutation by at least comparing an amino acid sequence of each input molecule of the pair of input molecules; and
identifying, based at least on the property difference between the pair of input molecules, that the mutation improves the value of the property.

57. The system of claim 56, wherein the identifying the one or more mutations further includes
applying the property computation model to determine a property difference between a different pair of input molecules,
identifying an additional mutation by at least comparing an amino acid sequence of each input molecule of the different pair of input molecules; and
identifying, based at least on the property difference between the different pair of input molecules, that the additional mutation also improves the value of the property.

58. The system of claim 57, wherein the operations further comprise:
generating an output molecule to include at least one of the mutation and the additional mutation.

59. The system of claim 57, wherein the mutation comprises a type of amino acid residue occupying a first position in the amino acid sequence of the first input molecule, and wherein the additional mutation comprises a same type of amino acid residue occupying a second position in the amino acid sequence of the first input molecule.

60. The system of claim 57, wherein the mutation comprises a position in the amino acid sequence of the first input molecule being occupied by a first type of amino acid residue, and wherein the additional mutation comprises a same position in the amino acid sequence of the first input molecule being occupied by a second type of amino acid residue.
